(19) United States
(12) Patent Application Publication
(10) Pub. No.: US 2023/0097992 A1
(43) Pub. Date: Mar. 30, 2023

(54) BIVALENT EGF FUSION TOXINS

(71) Applicant: The Regents of the University of Colorado, a body corporate, Denver, CO (US)

(72) Inventors: Zhirui WANG, Denver, CO (US); Shi-Long LU, Englewood, CO (US)

(21) Appl. No.: 17/907,264

(22) PCT Filed: Mar. 25, 2021
(86) PCT No.: PCT/US2021/024075

§ 371 Date: Sep. 25, 2022

Related U.S. Application Data
(60) Provisional application No. 62/994,615, filed on Mar. 25, 2020.

(51) Int. Cl. C07K 14/485; C07K 14/34; A61P 35/00

(52) U.S. Cl. CPC C07K 14/485; C07K 14/34; A61P 35/00; C07K 2319/00; A61K 38/00

(57) ABSTRACT

Bivalent epidermal growth factor (EGF) fusion toxin including two EGF domains are provided. The described fusion toxin demonstrates increased binding and cytotoxicity when compared to a fusion toxin having a single EGF binding domain (monovalent). Methods for treating epidermal growth factor receptor (EGFR)-positive cancers and related materials are also provided.

### CROSS-REFERENCE TO RELATED APPLICATION

This application claims the benefit of U.S. Provisional Application No. 62/994,615, entitled “BIVALENT EGF FUSION TOXINS,” filed Mar. 25, 2020, the disclosure of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Mar. 20, 2021, is named 517709_32_SL.txt and is 32,867 bytes in size.

### BACKGROUND

Head and neck cancers are fifth most common amongst all malignancies (>830,000 new cases per year) and are the sixth most common cause of cancer-related deaths worldwide (>430,000 deaths per year). About 65,000 new cases are diagnosed in the USA every year. Ninety percent of head and neck cancers are head and neck squamous cell carcinomas (HNSCCs). Treatment often involves surgery. Non-surgical treatment options include radiation therapy, chemotherapy (e.g., cisplatin/carboplatin and paclitaxel), targeted therapies (e.g., the monoclonal antibody cetuximab), and immunotherapy (e.g., blocking PD-1 and PD-L1 interaction using monoclonal antibodies pembrolizumab and nivolumab). While all these options are viable therapeutic strategies for treating HNSCC, the overall response rate and overall survival rate remains low.

Immunotoxins or fusion toxins (ITs) combine cell surface binding ligands or antibody-based single-chain fragment variable (scFv) with a peptide toxin. In cancer treatment, the ligand or scFv binds to a cell surface receptor expressed or overexpressed by malignant cells, and the toxin triggers cell death (Frankel et al., (2003) Semin Oncol. 30, 545-557). The diphtheria toxin (DT)-based monovalent human EGF fusion toxin (DAB₃₈₉EGF) was first studied in 1991 (Shaw et al., (1991) J Biol Chem. 266, 21118-21124) and evaluated for treating human glioblastoma multiforme cells and non-muscle-invasive urinary bladder cancer (Liu et al. (2003), Cancer Res. 63, 1834-1837); Yang et al., (2013) Clin Cancer Res. 19, 148-157). Further clinical development for systemic treatment was halted due to severe in vivo off-target toxicities caused by the monovalent human EGF fusion toxin (mono-EGF-IT).

New treatment option, particularly targeted therapies, are urgently needed; particularly for recurrent/metastatic (R/M) HNSCC patients.

### SUMMARY

In a first example (“Example 1”), a bivalent fusion toxin includes two epidermal growth factor (EGF) domains; a diphtheria toxin (DT) domain; and at least one linker.

In another example (“Example 2”), further to Example 1, the two epidermal growth factor domains each individually consist of human EGF or an EGFR-binding fragment thereof.

In another example (“Example 3”), further to Example 2, human EGF has an amino acid sequence having at least 95% identity to SEQ ID NO: 1.

In another example (“Example 4”), further to any one of Examples 1-3, the DT domain is selected from the group consisting of: DT₃₇₀ (SEQ ID NO: 3), DT₃₈₃ (SEQ ID NO: 4), DT₃₈₈ (SEQ ID NO: 5), DT₃₈₉ (SEQ ID NO: 6), and DT₃₉₀ (SEQ ID NO: 7).

In another example (“Example 5”), further to any one of Example 1-4, the DT domain is DT₃₉₀ (SEQ ID NO: 7).

In another example (“Example 6”), further to any one of Examples 1-5, the at least one linker includes at least one of G₄S (SEQ ID NO: 8) and (G₄S)₃ (SEQ ID NO: 9).

In another example (“Example 7”), further to any one of Examples 1-6, the bivalent fusion toxin includes a first linker between the DT domain and a first EGF domain, and a second linker between the first EGF domain and a second EGF domain.

In another example (“Example 8”), further to Example 7, the first linker is G₄S (SEQ ID NO: 8) and the second linker is (G₄S)₃ (SEQ ID NO: 9).

In another example (“Example 9”), further to Example 1, the bivalent fusion toxin comprises an amino acid sequence having at least 90% identity to SEQ ID NO: 10

In another example (“Example 10”), a nucleic acid molecule encodes the bivalent fusion toxin of any one of Examples 1-9.

In another example (“Example 11”), further to Example 10, the nucleic acid molecule is codon optimized.

In another example (“Example 12”), an expression vector includes the nucleic acid molecule of Example 10 or Example 11.

In another example (“Example 13”), an isolated cell includes the expression vector of Example 12

In another example (“Example 14”), a pharmaceutical composition includes the bivalent fusion toxin of any one of claims 1-9 and a pharmaceutically acceptable carrier.

In another example (“Example 15”), a method of treating an epidermal growth factor receptor (EGFR)-positive cancer in a patient in need thereof includes administering to the patient a therapeutically effective amount of the pharmaceutical composition of Example 14.

In another example (“Example 16”), further to Example 15, the EGFR-positive cancer is an EGFR-positive neck and head squamous cell carcinoma (HNSCC).

In another example (“Example 17”), further to Example 15 or Example 16, the EGFR-positive cancer is non-responsive to or recurring following standard of care treatment.

In another example (“Example 18”), further to Example 17, the EGFR-positive cancer is an EGFR-positive HNSCC and the standard of care treatment includes administration of erlotinib to the patient.

In another example (“Example 19”), further to any one of Examples 15-18, EGFRs of EGFR-positive cancer cells include at least one amino acid mutation.

In another example (“Example 20”), further to Example 19, the at least one amino acid mutation includes T790M.

### DESCRIPTION OF THE DRAWINGS

The accompanying drawings are incorporated into and form a part of the specification to illustrate support for and examples of the present disclosure. The drawings illustrate examples of the disclosure and are not to be construed as limiting the disclosure to the illustrated and described examples. Further features and advantages will become apparent from the following, more detailed, description of the various aspects, embodiments, and configurations of the disclosure.

FIG. 1 is a schematic diagram of a monovalent EGF-DT₃₉₀ fusion toxin (mEGF-IT) and a bivalent EGF-DT₃₉₀ fusion toxin (bEGF-IT) according to certain embodiments. G₄S is SEQ ID NO: 8. (G₄S)₃ is SEQ ID NO: 9.

FIGS. 2A-2C are representative photographs of SDA-PAGE and Western blot analysis of the mEGF-IT and bEGF-IT. FIG. 2A: SDS-PAGE analysis (4-12% NuPAGE, ThermoFisher Scientific).

FIG. 2B: Western blot analysis using a mouse anti-His mAb (clone #: 4A12E4, ThermoFisher Scientific). FIG. 2C: Western blot analysis using a mouse anti-diphtheria toxin mAb (clone #3B6, Meridian). FIGS. 2A-2C: Lane 1: Protein marker; Lane 2: mEGF-IT (50.1 kDa); Lane 3: bEGF-IT (57.2 kDa).

FIG. 3A presents representative data from flow cytometry binding affinity analysis of the mEGF-IT and bEGF-IT to human EGFR⁺ HNSCC Cal27 cells. Anti-human EGFR mAb was included as positive control, biotinylated anti-murine PD-1 toxin as a negative control for background due to protein biotinylation. The data are representative of three individual experiments.

FIG. 3B presents K_{D} determination data for the human EGF fusion toxins binding to Cal27 cells using flow cytometry and nonlinear least squares fit. Mean fluorescence intensity (MFI) was plotted over a wide range of concentrations of biotinylated mEGF-IT or bEGF-IT. The nonlinear regression fit presented was based on the equation Y=Bₘₐₓ*X/(K_{D}+X), where Y=MFI at the given biotinylated fusion toxin concentration after subtracting off the background; X=biotinylated fusion toxin concentration; Bₘₐₓ=the maximum specific binding in the same units as Y.

FIGS. 3C-D present representative data from analysis of the blocking of anti-human EGFR mAb binding to human EGFR-expressing (EGFR⁺) HNSCC Cal27 by mEGF-IT and bEGF-IT using flow cytometry. FIG. 3C presents representative flow cytometry data, and FIG. 3D presents the percentage inhibition of anti-human EGFR mAb binding to Cal27 cells plotted versus the concentration of binding competitor (mono-EGF-IT or bi-EGF-IT). The data are representative of three individual experiments.

FIG. 4 presents the data from in vitro efficacy analysis of mEGF-IT and bEGF-IT against human EGFR-positive HNSCC Cal27 cells using CellTiter-Glo® Luminescent Cell Viability Assay (Promega Corporation, Madison, Wis.). Y-axis: inhibition rate of the cell viability by determining the number of viable cells based on the quantification of the ATP present. X-axis: plated fusion toxin concentration. Cycloheximide (1.25 mg/mL) was used as a positive control. The negative control contained cells without fusion toxin. Data were from three individual experiments. Error bars indicate SD.

FIG. 5 presents the data from off-target analysis of the human EGF fusion toxins to three human EGFR-negative (EGFR⁻) tumor cell lines (JeKo-1, Jurkat and EL4). Human EGFR-positive (EGFR⁺) HNSCC SCC10B was included as positive control cell line. FIG. 5A: Flow cytometry binding analysis of the human EGF fusion toxins to three human EGFR⁻ tumor cell lines. The data are representative of three individual experiments. FIG. 5B: In vitro efficacy analysis of the human EGF fusion toxins to the three human EGFR⁻ tumor cell lines using CellTiter-Glo® Luminescent Cell Viability Assay. Data are representative of three individual assays. Error bars indicate SD.

FIGS. 6A-6D presents the data from in vivo efficacy analysis of the human EGF fusion toxins against subcutaneous xenografts in NSG mice from two animal studies. FIG. 6A depicts the survival curve for NSG mice injected with Cal27 cells. The Cal27 cells were subcutaneously (SQ) injected into the right flank of NSG mice (first cohort) followed by treatment with DT₃₉₀, mono-EGF-IT, bi-EGF-IT, or erlotinib once daily for 10 consecutive days beginning on day 4 after the tumor cell injection. Kaplan-Meier survival curves were recorded for the DT₃₉₀ (blue line), mono-EGF-IT (red line), bi-EGF-IT (green line), and erlotinib (purple line) groups. FIGS. 6B-D depict tumor volume and weight data from the second study performed with a second cohort of NSG mice. Cal27 cells were subcutaneously injected into the flanks of the second cohort of NSG mice that were then treated with DT₃₉₀, mono-EGF-IT, bi-EGF-IT, or erlotinib. Mice were euthanized on day 14 after tumor cell injection, when the first mouse in the DT390 group reached the end point. FIG. 6B: Tumor volumes were measured periodically, and the growth kinetics of the four groups were plotted. FIG. 6B depicts the tumor growth kinetics for a period of 14 days. FIG. 6C presents a bar graph representing the tumor weights at day 14. FIG. 6D depicts images of harvested tumors on day 14. Scale bar: 1 cm. FIGS. 6A-6C: *P<0.05; **P<0.01; ***P<0.001; ****P<0.0001; ns: not significant. The P-values for the survival curves in FIG. 6A were calculated using the Mantel-Cox log-rank test and that for the comparisons in FIGS. 6B and C were calculated using the two-tailed Student t-test (GraphPad Prism 9.0.0).

FIGS. 7A-7C presents the data from two animal studies of in vivo efficacy analysis of the human EGF fusion toxins against tongue squamous cell carcinomas (SCCs) in NSG mice. FIG. 7A presents the data from in vivo efficacy analysis of the human EGF fusion toxins using human EGFR-positive HNSCC Cal27 tongue-orthotopic tumor-bearing NSG mouse model. Eight million human EGFR-positive HNSCC Cal27 cells were injected into the tongue on day 0. Mice were injected intraperitoneally (IP) with the EGF fusion toxin or DT390 at 50 μg/kg, starting on day 4, once daily for 10 consecutive days. Erlotinib was administered via intragastric gavage at 20 mg/kg, starting on day 4, once daily for 10 consecutive days. The schedule of the fusion toxin and tumor cell injection is pictured in the schematic below the survival curve. The vertical arrows indicate the days on which the tumor cells or the fusion toxins were injected. *P<0.05; **P<0.01; ***P<0.001; ****P<0.0001; ns: not significant.

FIG. 7B presents the data from a further in vivo efficacy analysis of the human EGF fusion toxins. Cal27 cells were injected into the tongues of a second cohort of NSG mice that were then treated with DT390 (n=6), mono-EGF-IT (n=6), bi-EGF-IT (n=6), or erlotinib (n=6). The mice were euthanized on day 8 after tumor cell injection when the first mouse in the DT₃₉₀ group reached the end point. The photograph depicts tongue squamous cell carcinomas (SCCs) (circled by the black dotted lines). Scale bar: 1 cm. FIG. 7C presents the FIG. 7B tumor volume comparison of the treatment groups. *P<0.05; **P<0.01

FIG. 8A-C presents data from in vivo efficacy analysis of the human EGF fusion toxins using an experimental lung metastasis NSG mouse model. FIG. 8A presents data from a first animal study for survival curve analysis. NSG mice were IV injected with one million human EGFR⁺ HNSCC Cal27 cells on day 0. Mice were injected intraperitoneally (IP) with the EGF fusion toxin or DT390 at 50 μg/kg, once daily for 10 consecutive days starting on day 4 after the tumor cell injection. Kaplan-Meier survival curves were recorded for the DT390 (blue line), mono-EGF-IT (red line), bi-EGF-IT (green line), and erlotinib (purple line) groups. The schedule of the fusion toxin and tumor cell injection is pictured in the schematic below the survival curve. The vertical arrows indicate the days on which the tumor cells or the fusion toxins were injected. *P<0.05; **P<0.01; ***P<0.001; ****P<0.0001. The P-values for the survival curves were calculated using the Mantel-Cox log-rank test (GraphPad Prism9.0).

FIGS. 8B and C present data from a second animal study for a pathology analysis. Cal27 cells were intravenously injected into a second cohort of NSG mice that were then treated with DT390 (n=12), mono-EGF-IT (n=12), bi-EGF-IT (n=6), or erlotinib (n=6). The mice were euthanized on day 10 after tumor cell injection when the first mouse in the DT390 group reached the end point. FIG. 8B presents H&E pictures of lung metastases. Metastases are indicated in the upper panels by black arrowheads. Scale bar: 500 μm. The lower panels are higher magnification pictures of the area in black rectangles in the upper panels. FIG. 8C presents the data on the number of lung metastasis present in the DT390, mono-EGF-IT, bi-EGF-IT and erlotinib treatment groups. Error bars indicate standard deviation (SD).

FIG. 9 presents the data from in vitro efficacy analysis of the human EGF fusion toxins against human EGFR-positive HNSCC cell lines using CellTiter-Glo® Luminescent Cell Viability Assay (Promega) Y-axis: inhibition rate of the cell viability by determining the number of viable cells based on the quantification of the ATP present. X-axis: plated fusion toxin concentration. Cycloheximide (1.25 mg/mL) was used as a positive control. The negative control contained cells without fusion toxin. Data were from three individual experiments for each HNSCC cell line. Error bars indicate SD.

FIG. 10 presents the data from in vitro efficacy analysis of erlotinib against 14 human EGFR-positive HNSCC cell lines using CellTiter-Glo® Luminescent Cell Viability Assay (Promega). Y-axis: inhibition rate of the cell viability by determining the number of viable cells based on the quantification of the ATP present. X-axis: plated erlotinib concentration. Cycloheximide (1.25 mg/mL) was used as a positive control. The negative control contained cells without erlotinib. Data were from three individual experiments for each HNSCC cell line.

FIGS. 11A-11N present flow cytometry binding affinity analysis (top panel) and K_{D} determination (bottom panel) of the mEGF-IT and bEGF-IT to 13 human EGFR positive HNSCC cell lines: FIG. 11A) Fadu; FIG. 11B) HN6; FIG. 11C) UMCC1; FIG. 11D) UMSCC2; FIG. 11E) UMSCC10A; FIG. 11F) UMSCC10B; FIG. 11G) UMSCC11A; FIG. 11H) UMSCC22A; FIG. 11I) UMSCC22B; FIG. 11J) UMSCC47; FIG. 11K) Tu167; FIG. 11L) Vu1131; FIG. 11M) Vu1365. FIG. 11N depicts a K_{D} comparison between mono-EGF-IT and bi-EGF-IT in the 14 EGFR⁺ HNSCC cells lines. Anti-human EGFR mAb was included as positive control, biotinylated anti-murine PD-1 immunotoxin as a negative control for background due to protein biotinylation. The data were from three individual experiments. K_{D} was determined using flow cytometry and nonlinear least squares fit. Mean fluorescence intensity (MFI) was plotted over a wide range of concentrations of biotinylated mEGF-IT or bEGF-IT. The nonlinear regression fit shown was based on the equation Y=Bₘₐₓ*X/(K_{D}+X), where Y=MFI at the given biotinylated fusion toxin concentration after subtracting off the background; X=biotinylated fusion toxin concentration; Bₘₐₓ=the maximum specific binding in the same units as Y.

FIGS. 12A-C presents representative images of necropsy results for NSG mice treated with mono-EGF-IT or bi-EGF-IT without tumor cell injection. Mice were euthanized on day 13 after the first injection of the fusion toxins. The images show representative gross changes observed in the ear (FIG. 12A), liver (FIG. 12C) and gastrointestinal tract of a representative NSG mouse treated with mono-EGF-IT compared to a representative NSG mouse treated with bi-EGF-IT.

FIGS. 13A-C present in vitro binding affinity and efficacy analysis of the bEGF-IT to the residual Cal27 tumor cells. FIG. 13A: Flow cytometry binding affinity analysis of the bEGF-IT to the residual Cal27 tumor cells. The data were representative of three individual experiments. FIG. 13B: K_{D} determination of the bEGF-IT to the residual Cal27 tumor cells using flow cytometry and nonlinear least squares fit. FIG. 13C: In vitro efficacy analysis of the bEGF-IT to the residual Cal27 tumor cells using CellTiter-Glo® Luminescent Cell Viability Assay. bEGF-IT group (green line); Anti-murine PD-1 immunotoxin group as negative control (blue line). Data were from three individual experiments. Error bars indicate SD.

### DETAILED DESCRIPTION

In the following sections, various compositions and methods are described in order to detail various embodiments. Practicing the various embodiments does not require the employment of all of the specific details outlined herein, but rather concentrations, times, and other specific details may be modified. In some cases, well known methods or components have not been included in the description.

This disclosure relates to a new and improved therapeutic agent for the treatment of EGFR-expressing (EGFR⁺) HNSCC. The therapeutic agent created is a diphtheria toxin-based bivalent human epidermal growth factor fusion toxin (bi-EGF-IT). The bi-EGF-IT was tested for in vitro binding affinity, cytotoxicity, and specificity using 14 human EGFR⁺ HNSCC cell lines and three human EGFR-negative cancer cell lines. As disclosed herein, bi-EGF-IT had increased binding affinity for EGFR-expressing HNSCC compared with the monovalent version (mono-EGF-IT), and both versions specifically depleted EGFR⁺ HNSCC, but not EGFR-negative cell lines, in vitro. Bi-EGF-IT exhibited a comparable potency to that of the FDA-approved EGFR inhibitor, erlotinib, for inhibiting HNSCC tumor growth in vivo using both subcutaneous and orthotopic HNSCC xenograft mouse models. Moreover, when tested in an experimental metastasis model, survival was significantly longer in the bi-EGF-IT treatment group than the erlotinib treatment group, with a significantly reduced number of metastases compared with mono-EGF-IT. Furthermore, in vivo off-target toxicities were significantly reduced in the bi-EGF-IT treatment group compared with the mono-EGF-IT group. These results demonstrate that bi-EGF-IT is more effective and markedly less toxic at inhibiting primary HNSCC tumor growth and metastasis than mono-EGF-IT and erlotinib.

Accordingly, bivalent epidermal growth factor receptor (EGFR)-targeted toxins are disclosed and described herein. The disclosed bivalent epidermal growth factor (EGF) fusion toxins provide both improved efficacy and remarkably less in vivo off-target toxicity, relative to monovalent EGF fusion toxins. The targeted toxins described herein can be used in the treatment of EGFR-positive tumors.

### Definitions

As used herein, “treat” in reference to a condition means: (1) to ameliorate or prevent the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, and/or (4) to slow the progression of the condition or one or more of the biological manifestations of the condition.

As used herein, “therapeutically effective amount” in reference to an agent refers to a dose that is adequate to treat the subject's condition but low enough to avoid serious side effects at a reasonable benefit/risk ratio within the scope of sound medical judgment. The safe and effective amount of an agent will vary with the particular agent chosen (e.g. consider the potency, efficacy, and half-life of the compound); the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be determined by the skilled artisan.

For any compound, agent, or composition, the therapeutically effective amount can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

It will be appreciated by one of skill in the art that various diseases or disorders could require prolonged treatment involving multiple administrations, perhaps using the described bivalent EGF fusion toxins in each or various rounds of administration.

The amount or dose of the bivalent EGF fusion toxin administered should be sufficient to affect a therapeutic response in the subject over a reasonable time frame. The dose may be determined by the efficacy of the particular inventive fusion toxin and the condition of the subject, as well as the body weight of the subject to be treated.

A “subject” means any individual having, having symptoms of, or at risk for an EGFR-positive cancer. A subject may be human or non-human, and may include, for example, animals or species used as “model systems” for research purposes, such as a mouse model. In certain embodiments, the subject is a human patient having or at risk of developing an EGFR-positive cancer.

As used herein, a “pharmaceutical composition” is a formulation containing a compound or agent (e.g., bivalent EGF fusion toxin) in a form suitable for administration to a subject. Compounds and agents disclosed herein each can be formulated individually or in any combination into one or more pharmaceutical compositions. Accordingly, one or more administration routes can be properly elected based on the dosage form of each pharmaceutical composition. Alternatively, a compound or agent disclosed herein and one or more other therapeutic agents described herein can be formulated as one pharmaceutical composition.

### Bivalent EGF Fusion Toxins

Described herein are bivalent fusion toxins (i.e., fusion proteins) including two epidermal growth factor (EGF) domains and a cytotoxic domain or portion of a protein toxin. The two EGF domains target the cytotoxic domain to cells expressing the epidermal growth factor receptor (EGFR). EGFR overexpression and/or mutation occurs in many cancer types, including head and neck squamous cell carcinoma (HNSCC), brain cancer (e.g., glioblastoma multiforme), breast cancers, colorectal carcinomas, and lung cancers. For example, EGFR is highly expressed in up to 90% of HNSCCs. EGFR is a promising therapeutic target for EGFR-positive cancers, and indeed, diphtheria toxin-based EGF fusion toxins have been developed and studied for use in, for example, human glioblastoma multiforme cells and non-muscle-invasive urinary bladder cancer. As disclosed and described herein, the provided bivalent fusion toxins more effectively bind and kill cancer cells expressing EGFR than a monovalent fusion toxin having a single EGF domain.

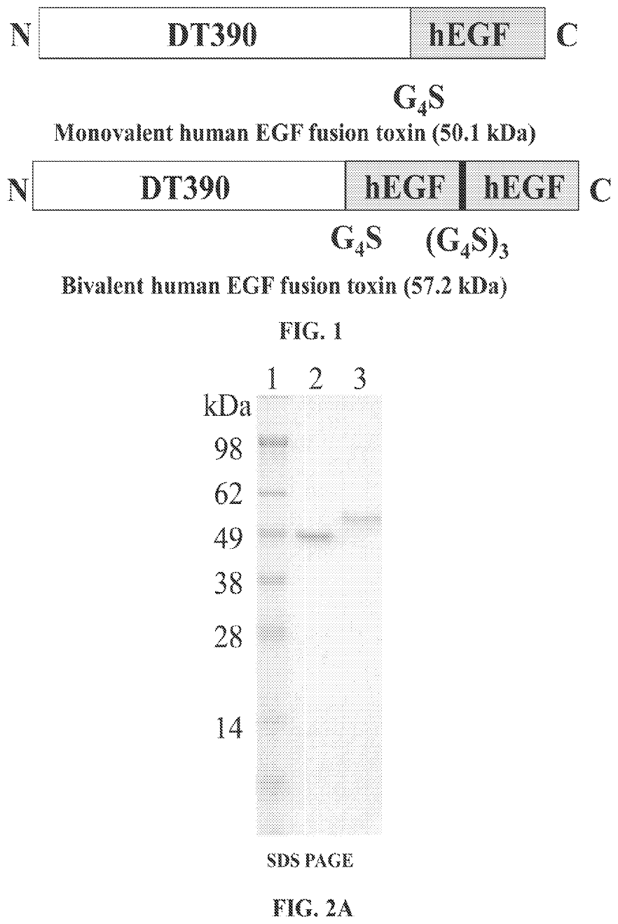

Bivalent fusion toxins of the present disclosure include two EGF domains, a cytotoxic domain, and at least one linker. A representative embodiment of a bivalent fusion toxin is provided in FIG. 1, which depicts a bivalent fusion toxin including a diphtheria toxin domain (DT₃₉₀) linked to a first EGF domain by a G₄S linker (SEQ ID NO: 8), with the first EGF domain being linked to a second EGF domain by a (G₄S)₃ (SEQ ID NO: 9) linker. The embodiment depicted by FIG. 1 is only one embodiment of the contemplated bivalent EGF fusion toxins. Other embodiments are also contemplated, as further described herein.

In some embodiments, the EGF domains are each a full-length EGF polypeptide. In other embodiments, the EGF domains are each EGFR-binding EGF fragments. If human EGFR is to be targeted, the EGF domains can be of human origin. In a particular embodiment, each of the EGF domains is full-length human EGF, which has the amino acid sequence of SEQ ID NO: 1. In some embodiments, each EGF domain has an amino acid sequence having at least 90%, at least 91%, at least 92%, and least 93%, at least 94%, at least 95%, at least 96%, at least 97%, at least 98%, at least 99%, or 100% identity to SEQ ID NO: 1.

In yet other embodiments, the EGF domains are full-length polypeptides or EGFR-binding fragments of a polypeptide belonging to the EGF-family/EGF-like family of polypeptides, which include heparin-binding EGF-like growth factor (HB-EGF), transforming growth factor-α (TGF-α), amphiregulin (AR), epiregulin (EPR), epigen, betacellulin (BTC), neuregulin-1 (NRG1), neuregulin-2 (NRG2), neuregulin-3 (NRG3), neuregulin-4 (NRG4).

In certain embodiments, the two EGF domains of the bivalent EGF fusion toxin are identical. It will be recognized that the EGF domains of a bivalent EGF fusion toxin need not be identical. For example, a full-length EGF polypeptide and an EGFR-binding fragment of EGF can be included in the fusion toxin. Similarly, a first EGF domain of the fusion toxin can be from one EGF-family/EGF-like family member, and a second EGF domain of the fusion toxin can be from a second (i.e., different) EGF-family/EGF-like family member. The two EGF domains of the bivalent EGF fusion toxin can be appropriately selected based on the target receptor(s).

The cytotoxic domain of the bivalent EGF fusion toxin can be any polypeptide domain or polypeptide fragment of a suitable protein toxin. Suitable protein toxins include, but are not limited to, diphtheria toxin (DT), Pseudomonas exotoxin (PE), and the ribosome-inactivating proteins (RIPs) (e.g., ricin, abrin, and saporins). Many protein toxins and cytotoxic fragments thereof have been incorporated into various fusion toxins, are known in the art, and can be selected for used in the present bivalent EGF fusion toxins based on, for example, the target cell, expression system, etc.

In some embodiments, cytotoxic domain is a DT domain. DT is a 535 amino acid residue with three domains. The C-terminal domain (a.a. 390-535) has a β-sheet-rich tertiary structure and functions to bind the protein toxin to the cell surface HB-EGF receptor (HB-EGFR). The central domain (a.a. 201-389) is rich in amphipathic α-helices and facilitates translocation of the catalytic domain to the cytosol. The N-terminal domain (a.a. 1-200) is a dADP-ribosylase and catalytically adds ADP to the dipthamide residue of elongation factor 2 (EF2), leading to inactivation of protein synthesis. The DT amino acid sequence is presented in SEQ ID NO: 2. The C-terminal receptor-binding domain is often removed to form fusion toxins. Several DT iterations have been developed for use in fusion toxins, including, for example, DT₃₇₀ (SEQ ID NO: 3), DT₃₈₃ (SEQ ID NO: 4), DT₃₈₈ (SEQ ID NO: 5), DT₃₈₉ (SEQ ID NO: 6), and DT₃₉₀ (SEQ ID NO: 7). Any of these DT fragments can be used in the described bivalent EGF fusion toxins. In particular embodiments, the cytotoxic domain of the bivalent EGF fusion toxin is DT₃₉₀ (SEQ ID NO: 7).

Many different linkers are known in the art and can be used to link together the various domains of the bivalent EGF fusion toxin. Appropriate linkers can be selected to achieve the desired flexibility within the fusion toxins; the desired toxicity, as linker length and sequence have been demonstrated to affect toxicity, and fusion toxin expression. In some embodiments, a bivalent EGF fusion toxin includes at least two linkers. In some embodiments, the linkers can be (G₄S)ₙ, where n=1-5 (SEQ ID NO: 16). In certain embodiments, the bivalent EGF fusion toxin includes at least one of G₄S (GGGGS; SEQ ID NO: 8) and (G₄S)₃ (GGGGSGGGGSGGGGS; SEQ ID NO: 9).

The cytotoxic domain can be linked to the first EGF domain either directly or via a linker. In some embodiments, the cytotoxic domain (e.g., DT₃₉₀) is linked to the first EGF domain via the linker G₄S (SEQ ID NO: 8). Generally, the first EGF domain is linked to the second EGF domain via a linker to provide some degree of flexibility between the two EGF domains. In certain embodiments, the first EGF domain is linked to the second EGF domain via the linker (G₄S)₃ (SEQ ID NO: 9).

In certain embodiments, the bivalent EGF fusion toxin is arranged as illustrated in FIG. 1, with DT₃₉₀ being linked to a first full-length EGF domain via G₄S (SEQ ID NO: 8), and the first full-length EGF domain linked to a second full-length EGF domain via (G₄S)₃ (SEQ ID NO: 9) In some embodiments, such a fusion toxin includes or has an amino acid sequence at least 90% identity to SEQ ID NO: 10.

### Nucleic Acid Molecules and Expression Vectors

Also provided herein are nucleic acid molecules comprising a nucleotide sequence encoding any of the bivalent EGF fusion toxins described herein, or individual domains thereof. Such nucleic acid molecules can be designed and prepared using known sequences and standard genetic engineering techniques. Nucleic acid molecules encoding cytotoxin domains of protein toxins and EGF domains can be obtained by cloning from available genomic or cDNA libraries with the use of probes based on known nucleic acid or amino acid sequences, by polymerase chain reaction (PCR), or by direct synthesis. In certain embodiments, the nucleic acid molecules encoding the bivalent EGF fusion toxins are codon optimized.

In certain embodiments, the nucleic acid molecules disclosed herein are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

This disclosure also provides a nucleic acid molecule comprising a nucleotide sequence that is at least about 90% or more, e.g., about 91%, about 92%, about 93%, about 94%, about 95%, about 96%, about 97%, about 98%, or about 99% identical to any of the nucleic acids described herein.

In certain embodiments, the nucleic acid molecules of the present disclosure can be incorporated into an expression vector. In this regard, some embodiments provide recombinant expression vectors comprising any of the nucleic acid molecules of the disclosure. The term “recombinant expression vector” refers to a genetically-modified oligonucleotide or polynucleotide construct that permits the expression of an mRNA, protein, polypeptide, or peptide by a host cell, when the construct comprises a nucleotide sequence or expression construct encoding the mRNA, protein, polypeptide, or peptide, and the vector is contacted with the host cell under conditions sufficient to have the mRNA, protein, polypeptide, or peptide expressed within the cell. The vectors of the present disclosure are not naturally-occurring as a whole. However, parts of the vectors can be naturally occurring. The recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. In some embodiments, the non-naturally occurring or altered nucleotides or internucleotide linkages do not hinder the transcription or replication of the vector.

In certain embodiments, the recombinant expression vectors described herein comprise a suitable recombinant expression vector backbone and can be used to transform or transfect any suitable host cell. Suitable vector backbones include those designed for propagation and expansion or for expression or both, such as plasmids and viruses. The vector backbones can be selected from the group of the pwPICZalspha vector series, the pUC vector series (Fermentas Life Sciences, Glen Burnie, Md.), the pBluescript vector series (Stratagene, LaJolla, Calif.), the pET vector series (Novagen, Madison, Wis.), the pGEX vector series (Pharmacia Biotech, Uppsala, Sweden), and the pEX vector series (Clontech, Palo Alto, Calif.). Bacteriophage vector backboness, such as, GTlO, GTll, Zapll (Stratagene), EMBL4, and NM1149 also can be used. Examples of animal expression vectors include pEUK-Cl, pMAM, and pMAMneo (Clontech). The recombinant expression vector may be a viral vector, e.g., a retroviral vector or a lentiviral vector. In particular embodiments, the vector backbone is the pwPICZalpha-DT390 vector.

In some embodiments, the recombinant expression vectors described herein can be prepared using recombinant DNA techniques described in the art. Constructs of expression vectors, which are circular or linear, can be prepared to contain a replication system functional in a prokaryotic or eukaryotic host cell.

The recombinant expression vector may comprise regulatory sequences, such as transcription and translation initiation and termination codons, which are specific to the type of host cell (e.g., yeast, bacterium, fungus, plant, or animal) into which the vector is to be introduced, as appropriate, and taking into consideration whether the vector is DNA- or RNA-based. The recombinant expression vector may also comprise restriction sites to facilitate cloning.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G4 18 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the CAR (including functional portions and functional variants thereof), or to the nucleotide sequence which is complementary to or which hybridizes to the nucleotide sequence encoding the CAR. The promoter can be a non-viral promoter or a viral promoter, e.g., a cytomegalovirus (CMV) promoter, an SV 40 promoter, an RSV promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Included in the scope of the present disclosure are conjugates, e.g., bioconjugates, comprising any of the inventive bivalent EGF fusion toxins, nucleic acids, recombinant expression vectors, host cells, or populations of host cells.

An embodiment of the disclosure further provides a host cell comprising any of the recombinant expression vectors described herein. As used herein, the term “host cell” refers to any type of cell that can contain and express a recombinant expression vector described herein. The host cell can be a eukaryotic cell, e.g., yeast, plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells include, for example, Pichia pastoris cells. For purposes of amplifying or replicating the recombinant expression vector, the host cell may be a prokaryotic cell, e.g., a DH5a cell. For purposes of producing a fusion toxin, the host cell can be a yeast cell. While the host cell can be of any cell type, can originate from any type of tissue, and can be of any developmental stage, the host cell can be Pichia pastoris cell.

Also provided herein is a population of cells comprising at least one host cell described herein. The population of cells can be a heterogeneous population comprising the host cell comprising any of the recombinant expression vectors described, in addition to at least one other cell which does not comprise any of the recombinant expression vectors. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the disclosure, the population of cells is a clonal population comprising host cells comprising a recombinant expression vector as described herein.

A host cell-vector system used for replication of the expression vector and a host-vector system used for expression of the bivalent EGF fusion toxin can be as appropriate from among known systems of eukaryotic cells (e.g., mammalian cells, plant cells, yeast, insect cells) and prokaryotic cells (e.g., bacteria). As will be recognized by the skilled artisan, expression of the bivalent EGF fusion toxins can be complicated by the presence of the cytotoxic domain, which has the potential to kill the host cell. Expression systems capable of expressing such fusion toxins are known. For example, issues are known to arise when attempting to express DT-based fusion toxins in E. coli. A Pichia pastoris expression system was thus developed that is capable of expressing such DT-based fusion toxins (Liu et al. (2003), Protein Expr. Purif. 30, 262-274, which is hereby incorporated by reference in its entirety; see also U.S. Pat. No. 7,892,786, which is hereby incorporated by reference in its entirety).

The bivalent EGF fusion toxins can be isolated and/or purified. The term “isolated” as used herein means having been removed from its natural environment. The term “purified” or “isolated” does not require absolute purity or isolation; rather, it is intended as a relative term. Thus, for example, a purified (or isolated) host cell preparation is one in which the host cell is more pure than cells in their natural environment within the body. In some embodiments, a preparation of a host cell is purified such that the host cell represents at least about 50%, for example at least about 70%, of the total cell content of the preparation. For example, the purity can be at least about 50%, can be greater than about 60%, about 70% or about 80%, or can be about 100%.

### Pharmaceutical Compositions

The bivalent EGF fusion toxins can be formulated into a composition, such as a pharmaceutical composition. In this regard, an embodiment of the disclosure provides a pharmaceutical composition including any of the bivalent EGF fusion toxins described herein and a pharmaceutically acceptable carrier. The pharmaceutical compositions containing any of the fusion toxins can include more than one type of fusion toxin. In some embodiments, the pharmaceutical composition can also include one or more additional pharmaceutically active agents or drugs active against the cell(s) being targeted by the bivalent EGF fusion toxin. For example, one or more chemotherapeutic drugs can be included in the pharmaceutical composition. Suitable additional pharmaceutically active agents or drugs include, for example, tyrosine kinase inhibitors such as erlotinib, axitinib, dasatinib, imatinib, nilotinib, pazopanib, and sunitinib; bleomycin sulfate; cetuximab; docetaxel; hydroxyurea; pembrolizumab; methotrexate; and nivolumab for the treatment of HNSCC.

A pharmaceutically acceptable carrier can be any of those conventionally used and is limited only by chemico-physical considerations, such as solubility and lack of reactivity with the active agent(s), and by the route of administration. The pharmaceutically acceptable carriers described herein include, for example, vehicles, adjuvants, excipients, and diluents. It is preferred that the pharmaceutically acceptable carrier be one which has no detrimental side effects or toxicity under the conditions of use.

The choice of carrier will be determined in part by the particular bivalent EGF fusion toxin to be included, as well as by the particular method used to administer the fusion toxin. Accordingly, there are a variety of suitable formulations of the pharmaceutical composition of the present disclosure.

### Methods of Treatment

The bivalent EGF fusion toxins and pharmaceutical compositions including the bivalent EGF fusion toxins can be used in the treatment of EGFR-positive cancers. EGFR-positive cancers include, for example, many head and neck squamous cell carcinomas (HNSCC), certain brain cancers (e.g., glioblastoma multiforme), certain breast cancers, many colorectal carcinomas, and certain lung cancers. In certain embodiments, the EGF fusion toxins and pharmaceutical compositions can be used to treat HNSCC. In some embodiments, the EGFR-positive cancer to be treated is non-responsive standard of care treatment, or recurs following standard of care treatment. In some embodiments, the EGFR-positive cancer is an EGFR-positive HNSCC, and the standard of care treatment includes administration of erlotinib to the patient.

In certain embodiment, the bivalent EGF fusion toxins and pharmaceutical compositions of the present disclosure can be used in the treatment of tumors that intrinsically resistant or have acquired resistance to available anti-EGFR targeted therapy. Resistance mechanisms to anti-EGFR monoclonal antibodies and anti-EGFR tyrosine kinase inhibitors are known, and include, for example, overexpression of EGFR, dysregulation of EGFR internalization and degradation by ubiquitination, EGFRvIII expression, KRAS mutations, PTEN mutation/loss, increased heterodimerization of EGFR or HER2 with HER3, crosstalk (e.g., with HGF-MET or VEGF-VEGFR1), EGFR mutations (e.g., T790M, C797S), and histologic transformation (e.g., non-small cell lung cancer to small cell lung cancer). The bivalent EGF fusion toxins described herein may overcome these resistance mechanisms, as it is believed, without being held to theory, that the bivalent EGF fusion toxin functions through a different mechanism. For example, in certain embodiments, the bivalent EGF fusion toxins can be used in the treatment of EGFR-positive cancers in which the cancer cells express a mutated EGFR. Mutations occur in EGFR of many cancer cells, including but not limited to lung adenocarcinoma, glioblastoma multiforme, colon adenocarcinoma, non-small cell lung carcinoma, and HNSCC. In certain embodiments, the EGFR mutation is T790M, although other mutations are also contemplated. In the example of T790M, the mutation increases the receptor's affinity for ATP at the EGFR kinase domain, resulting in tyrosine kinase inhibitors (e.g., erlotinib) being outcompeted. The high affinity binding of the bivalent EGF fusion toxins can effectively target such mutated EGFRs, as its cytotoxic effect is independent of binding to the EGFR kinase domain.

As demonstrated herein, the bivalent EGF fusion toxins have improved affinity for EGFR relative to the monovalent EGF fusion toxins. In some embodiments, the high affinity binding of the bivalent EGF fusion toxins described herein can target tumors with low EGFR copy numbers.

Methods of treating an EGFR-positive cancer in a patient in need thereof include administering to the patient a therapeutically effective amount pharmaceutical composition that includes bivalent EGF fusion toxin.

The bivalent EGF fusion toxins may be administered in any suitable manner. In some embodiments, the fusion toxins are administered by injection (e.g., subcutaneously, intravenously, intratumorally, intraarterially, intramuscularly, intradermally, interperitoneally, or intrathecally). In particular embodiments, the fusion toxins are administered intravenously. A suitable pharmaceutically acceptable carrier for the fusion toxins for injection may include any isotonic carrier such as, for example, normal saline (about 0.90% w/v of NaCl in water, about 300 mOsm/L NaCl in water, or about 9.0 g NaCl per liter of water), NORMOSOL-R electrolyte solution (Abbott, Chicago, Ill.), PLASMA-LYTE A (Baxter, Deerfield, Ill.), about 5% dextrose in water, or Ringer's lactate. In an embodiment, the pharmaceutically acceptable carrier is supplemented with human serum albumen.

In some embodiments, the bivalent EGF fusion toxin can be administered with one or more additional therapeutic agents. When the bivalent EGF fusion toxin is to be administered with one or more additional therapeutic agents, one or more additional therapeutic agents can be co-administered to the subject. “Co-administer” and derivatives thereof mean administering one or more additional therapeutic agents and the inventive fusion toxin sufficiently close in time such that the fusion toxin can enhance the effect of the one or more additional therapeutic agents, or vice versa. In this regard, the fusion toxins can be administered first, and the one or more additional therapeutics can be administered second, or vice versa. Alternatively, the fusion toxin and the one or more additional therapeutic agents can be administered simultaneously.

### EXAMPLES

The materials, methods, and embodiments described herein are further defined in the following Examples. Certain embodiments are defined in the Examples herein. It should be understood that these Examples, while indicating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

### Cell Lines and Antibodies

HNSCC tumor cell lines used in the studies describe in these Experimental Examples are listed in Table 1. Human EGFR⁻ tumor cell lines Jeko-1 (ATCC® CRL-3006™), Jurkat (clone E6-1, ATCC® TIB-152™) and EL4 (ATCC® TIB-39™) were obtained from ATCC (Manassas, Va.). All mAbs used in the studies described in these Experimental Examples are listed in Table 2.

TABLE 1
HNSCC cell lines

| | | | | | p 53 | | | | |
|---|---|---|---|---|---|---|---|---|---|
| Cell Line | Location | Gender | Age | HPV | mutation | Fanconi | TNM | Stage | Source |
| Cal27 | tongue | male | 56 | | His193Leu | | | | ATCC |
| Fadu | hypopharynx | male | 56 | | Arg248Leu | | | | ATCC |
| HN6 | tongue | male | 54 | | | | T2N0M0 | | CU-AJ |
| UMSCC-1 | floor of | male | 72 | negative | no | | T2NoMo | II | UM |
| | mouth | | | | | | | | |
| UMSCC-2 | alveolar ridge | female | 64 | | | | T2NoMo | II | UM |
| UMSCC-10A | larynx (true | male | 57 | | Gly245Cys | | T3N0M0 | III | UM |
| | vocal cord) | | | | | | | | |
| UMSCC-10B | larynx-neck | male | 58 | | Gly245Cys | | T3N1M0 | III | UM |
| | lymph node | | | | | | | | |
| UMSCC-11A | larynx | male | 64 | | no | | | | UM |
| UMSCC-11B | larynx-neck | male | 64 | | | | | | UM |
| | lymph node | | | | | | | | |
| UMSCC-22A | hypopharynx | female | 59 | negative | Tyr220Cys | | T2N1M0 | III | UM |
| | hypopharynx- | | | | | | | | |
| | neck lymph | | | | | | | | |
| UMSCC-22B | node | female | 59 | negative | Tyr220Cys | | T2N1M0 | III | UM |
| UMSCC-47 | tongue | male | 53 | positive | no | | | | UM |
| Tu167 | floor of | male | 72 | | | | | | CU-AJ |
| | mouth | | | | | | | | |
| Vu1131 | floor of | female | 34 | negative | R273L | FA-C, | T4N2b | | Netherland |
| | mouth | | | | | c.67delG | | | |
| Vu1365 | mouth | male | 22 | negative | R282W | FA-A | | | Netherland |

CU: University of Colorado;
UM: University of Michigan.

TABLE 2
Antibodies

| Antibody Name | Clone# | Source | Cat# |
|---|---|---|---|
| PE-mouse anti-human EGFR | EGFR.1 | BD | 555997 |
| PE-streptavidin | L200 | BioLegend | 405204 |
| PE Mouse IgG2b, κ Isotype Ctrl Antibody | MPC-11 | BioLegend | 400313 |
| 7-Aminoactinomycin (7-AAD) | SP-34-2 | Sigma | A9400 |
| His Tag Antibody (Mouse) | 6G2A9 | Genscript | A00186 |
| Diphtheria Toxin Antibody (Mouse) | | Meridian Life Science | C86036M |
| Rat anti-Mouse IgG (H + L) Secondary | LO-MG-7 | Invitrogen | 04-6020 |
| Antibody, HRP | | | |

### DNA Construction

As illustrated in FIG. 1, human EGF fusion toxins include two domains: a DT390 domain (Woo et al. (2002), Protein Expr. Purif. 25, 270-82), which is hereby incorporated by reference in its entirety) and human EGF domain. A linker of four glycines and a serine residue (G₄S) (SEQ ID NO: 8) connected the DT390 domain to the human EGF domain. The two human EGF subunits of the human EGF domain of the bivalent EGF immunotoxin (also referred to herein as bEGF-IT; b-EGF-IT; and bi-EGF-IT) were joined by three tandem G₄S linkers (G₄S)₃ (SEQ ID NO: 9). Six histidines (6×His tag; SEQ ID NO: 17) were added to the C-terminus of each construct to facilitate purification.

mEGF-IT DNA construction. Codon-optimized human EGF DNA was cloned into pwPICZalpha-DT390 vector (Wang et al. (2015), Mol. Oncol. 9, 1458-1470, which is hereby incorporated by reference in its entirety) between NcoI and EcoRI sites, yielding the monovalent EGF toxin (also referred to herein as mEGF-IT; m-EGF-IT; and mono-EGF-IT) DNA construct.

bEGF-IT DNA construction. To prepare the first of two human EGF inserts, human EGF DNA was amplified using PCR primers hEGF-Nco carrying NcoI site and hEGF-Bam1 carrying BamHI site. The mEGF-IT DNA construct was used as the PCR template. The amplified PCR product was separated using DNA agarose gel electrophoresis. The expected PCR product band was cut out and extracted using QIAquick Gel Extraction Kit. The extracted DNA was digested using NcoI and BamHI and cleaned using QIAquick PCR Purification Kit to produce the first human EGF insert (Insert I). To prepare the second human EGF insert, human EGF DNA was amplified using hEGF-Bam2 carrying BamHI site and hEGF-Eco carrying EcoRI site. The mEGF-IT DNA construct was used as the PCR template. The amplified PCR product was separated with DNA agarose gel electrophoresis. The expected PCR product band was cut out and extracted using QIAquick Gel Extraction Kit. The extracted DNA was digested using BamHI and EcoRI and cleaned using QIAquick PCR Purification Kit to produce the second human EGF insert (Insert II). Insert I and Insert II (NcoI-EGF-BamHI-EGF-EcoRI) were together cloned into pwPICZalpha-DT390 vector between NcoI and EcoRI yielding the bEGF-IT DNA construct following sequence confirmation. All PCR primers used in the studies describe in these Experimental Examples are listed in Table 3. M-EGF-IT or bEGF-IT DNA construct was linearized using Sac I and transformed into diphtheria toxin resistant yeast Pichia pastoris strain (Liu et al., 2003). Both mEGF-IT and bEGF-IT were expressed and purified as described previously (Wang et al., 2015). Anti-murine PD-1 toxin was used as negative control for in vitro efficacy analysis and DT390 was used as negative control for in vivo efficacy analysis. DT390 and anti-murine PD-1 toxin were also expressed and purified using the same diphtheria toxin-resistant yeast Pichia pastoris expression system. Western blot analysis, flow cytometry binding affinity analysis and K_{D} determination were all performed as previously described (Wang et al., 2015).

TABLE 3
PCR primers (in order of appearance,
SEQ ID NOS: 11-14)

```
hEGF-Nco
5′ C ATG CCA TGG GGT GGT GGT GGT TCT AAC
                    NcoI
TCT GAC TCC GAG TGT CCA 3′
hEGF-Bam1
5′ CGG GGA TCC ACC ACC ACC AGA ACC ACC
                BamHI
ACC ACC TCT CAA CTC CCA CCA CTT CAA 3′
hEGF-Bam1
5′ CGG GGA TCC GGT GGT GGT GGT TCT AAC
                BamHI
TCT GAC TCC GAG TGT CCA 3′
hEGF-Eco
5′ CCG GAA TTC TTA GTG GTG GTG GTG GTG
                  EcoRI
GTG TCT CAA CTC CCA CCA CTT CAA 3′
```

### In Vitro Efficacy Analysis

In vitro efficacy analysis of the human EGF fusion toxins (mEGF-IT and bEGF-IT) to the human HNSCC tumor cell lines was performed using CellTiter-Glo® Luminescent Cell Viability Assay (Promega Corporation, Madison, Wis.) as described previously (Zheng et al. (2017), Mol. Oncol. 11, 584-594, which is hereby incorporated by reference in its entirety). This cell viability assay measures the luminescence produced because of ATP production from metabolically active cells. The increasing concentrations of the human EGF fusion toxins lead to cells death and a corresponding reduction in ATP related fluorescence. The luminescence signals were recorded using BioTek Synergy LX Multi-Mode Reader.

### In Vivo Efficacy Studies

HNSCC tumor cells were injected into six- to eight-week old immunodeficient NSG mice, which were divided into four groups for each tumor-bearing NSG mouse model: 1) DT390 as negative control group; 2) mEGF-IT group; 3) bEGF-IT group; and 4) Erlotinib (Tarceva, an EGFR-tyrosine kinase inhibitor) group as positive control.

HNSCC tumor cells were injected via the following routes: 1) Flank-xenograft solid tumor model: eight million human HNSCC Cal27 tumor cells were subcutaneously injected into the right flank at day 0. The tumor-bearing mice were randomly divided into the four experimental groups when the tumors were palpable at day 3. The tumor volume was measured using digital vernier calipers every 3 days and calculated according to the formula: volume (mm³)=(length×width×width)/2. 2) Tongue-orthotopic solid tumor model: eight million human HNSCC Cal27 tumor cells in 50 μl DMEM were injected into the tongue at day 0 following isoflurane anesthesia and the tumor-bearing mice were randomly divided into the planned four experimental groups at day 3. 3) Experimental metastasis model: one million human HNSCC Cal27 tumor cells in 200 μl DMEM were intravenously injected at day 0 via tail vein and the tumor-bearing mice were randomly divided into the four experimental groups at day 3.

Mono-EGF-IT (mEGF-IT), bEGF-IT, or DT₃₉₀ was intraperitoneally injected (IP) starting at day 4 at 50 μg/kg, once daily for 10 consecutive days. Positive control Erlotinib was administered via intragastric gavage at 20 mg/kg once daily for 10 consecutive days. The injected animals were observed daily for signs and symptoms of illness and scored at least twice weekly based on the parameters as previously reported (Peraino et al. (2013), J. Immunol. Methods 398-399, 33-43, which is hereby incorporated by reference in its entirety; Wang et al., 2015). The animals were humanely euthanized when the score exceeded the limit or the animal lost more than 15% of its pre-injection body weight. In addition, to assess whether the fusion toxins are toxic to the animals, NSG mice injected with the fusion toxins only were also included as control.

Culturing Residual Tumor Cells from Cal27 Tumor-Bearing NSG Mice

The tumor was removed from the sacrificed flank-xenograft solid tumor-bearing mice and washed with PBS twice in sterile manner. Then the tumor sample was cut into small pieces (approximately 1 mm³) on the lid of a petri dish and washed with PBS. The tissue pellet was resuspended in DMEM tissue culture medium and cultured in the T25 flask. After several days of culture, the tissue residues were removed and the residual tumor cells were cultured as parent Cal27 tumor cell line.

### Statistical Analysis

IC₅₀ was determined using nonlinear regression (curve fit) of Prism. For survival curve analysis, p values were calculated using log-rank (Mantel-Cox) test of Prism. For other statistical significance analysis, p values were calculated using Student's t-test of Prism. P<0.05 was considered as significant.

### Experimental Example 1—Construction and Expression of the Monovalent and Bivalent Human EGF Fusion Toxins

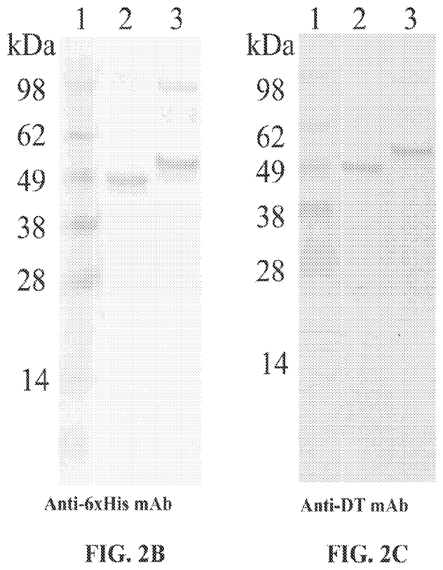

Codon-optimized human EGF DNA was synthesized and cloned into the truncated diphtheria toxin DT390-containing expression vector pwPICZalpha-DT390 as previously described (FIG. 1, Wang et al., 2015). Both mEGF-IT and bEGF-IT were expressed and purified using a diphtheria toxin-resistant Pichia pastoris expression system (Liu et al., 2003) as previously described (Wang et al., 2015). The final purification yield was −14 mg and −10 mg per liter of the original harvested supernatant for the monovalent and bivalent toxins, respectively. The purified EGF fusion toxins were analyzed using SDS-PAGE and Western blot, with an expected molecular weight of 50.1 kDa for the monovalent version and 57.2 kDa for the bivalent version (FIGS. 2A-2C).

### Experimental Example 2—In Vitro Binding Affinity and Efficacy Analysis of the Human EGF Fusion Toxins

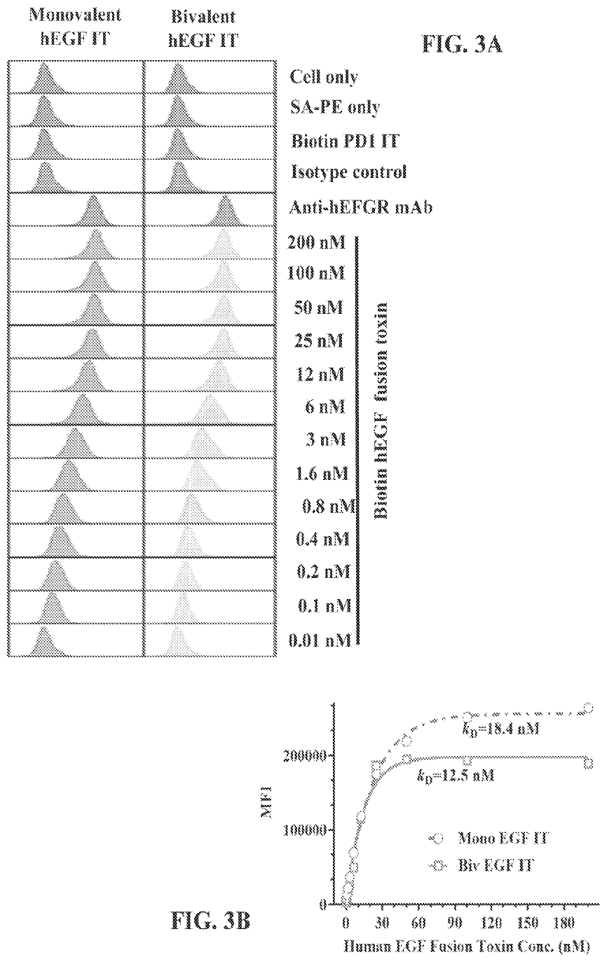

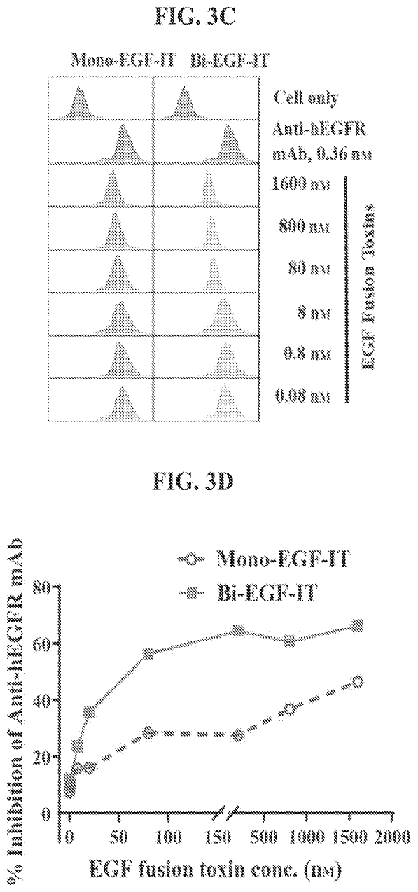

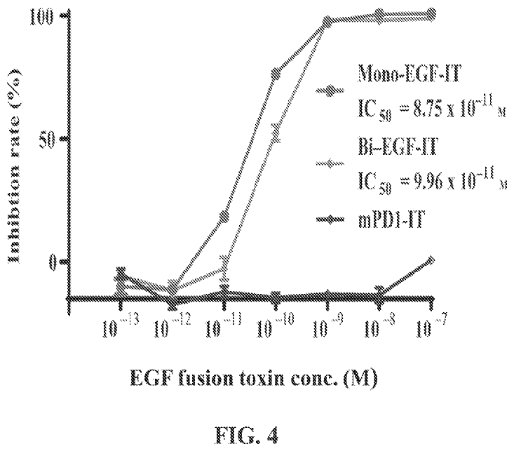

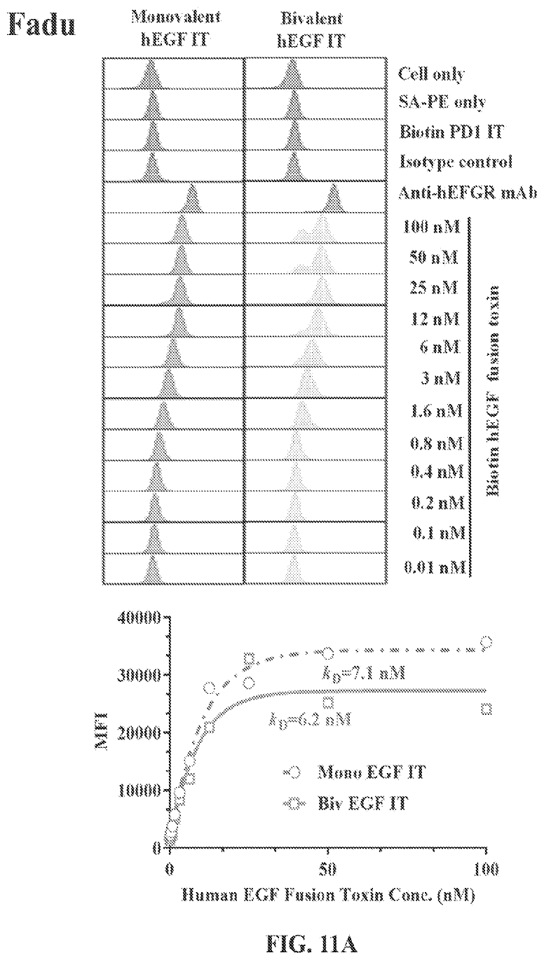

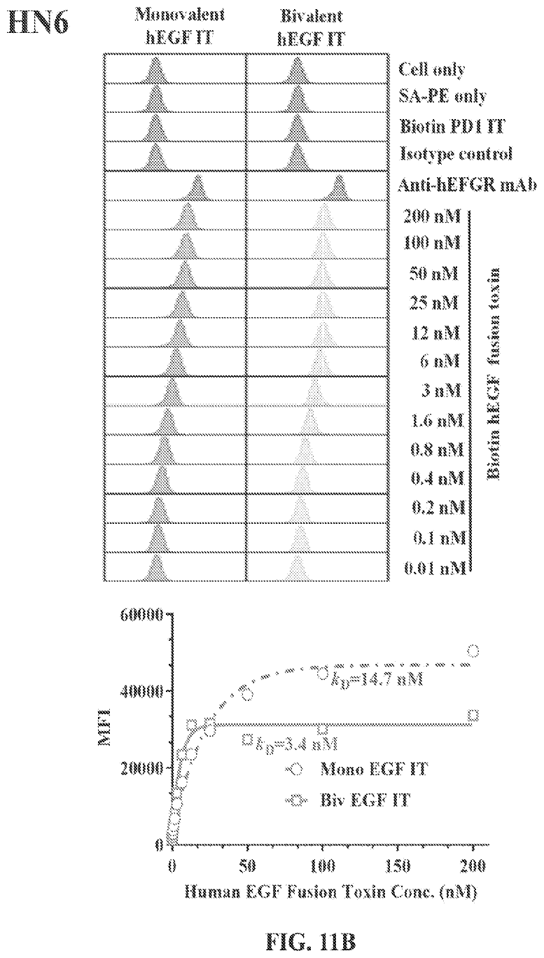

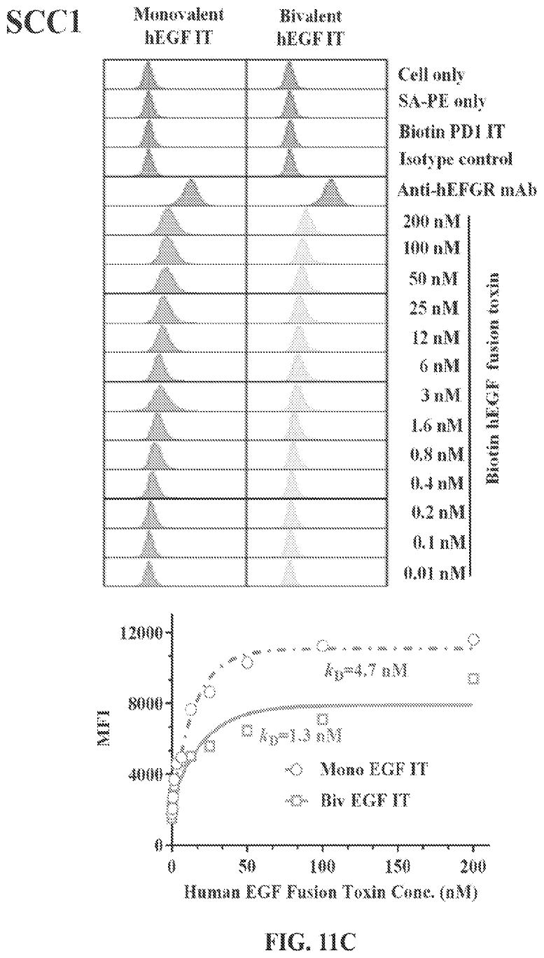

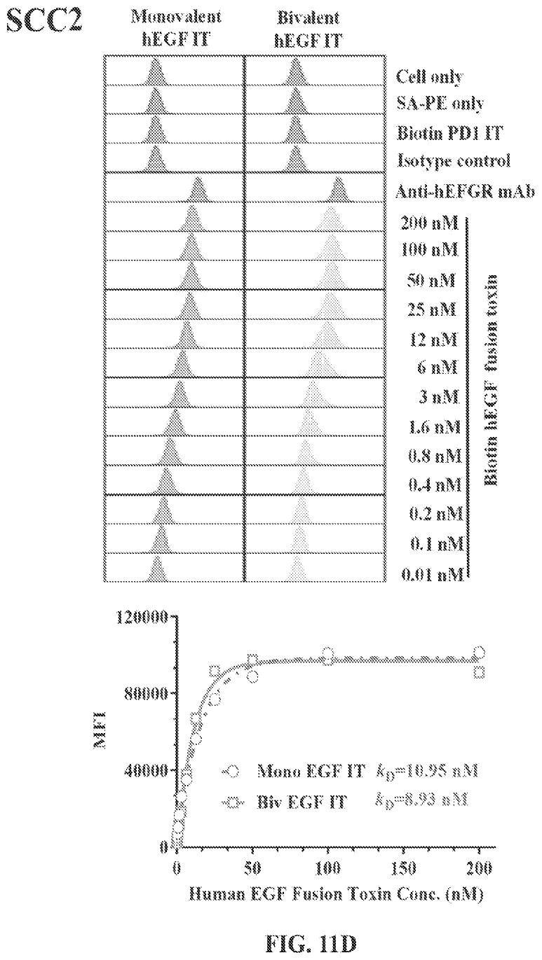

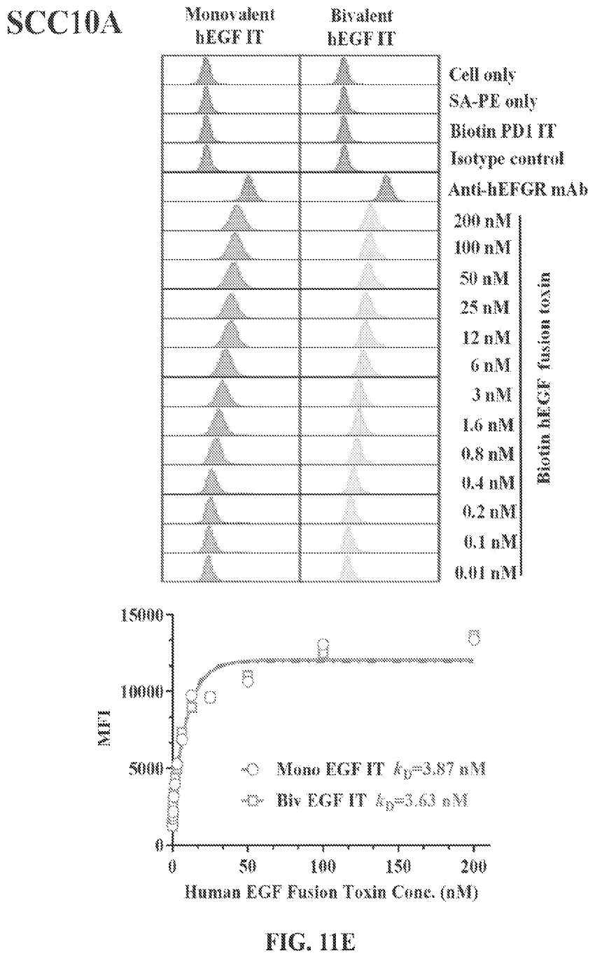

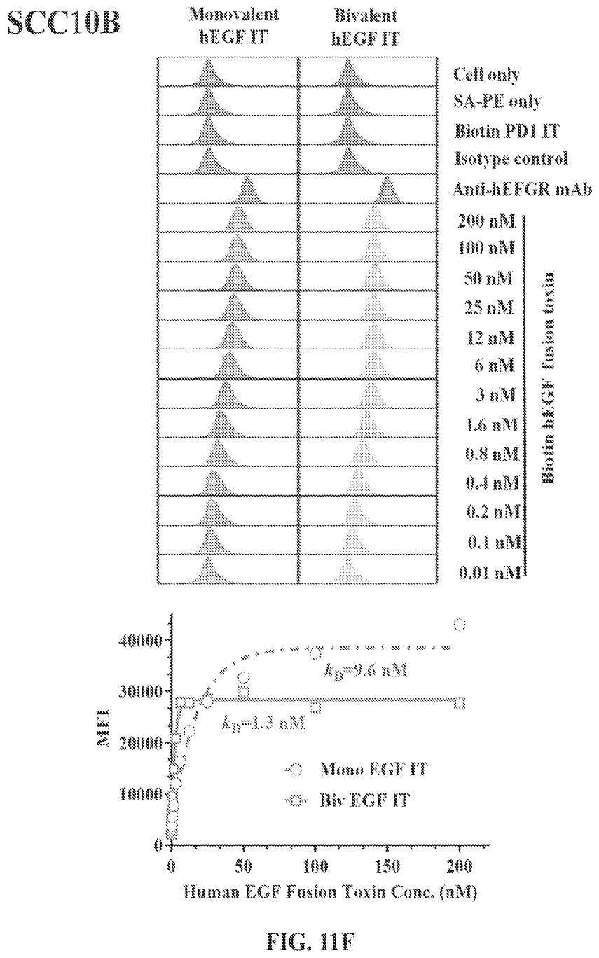

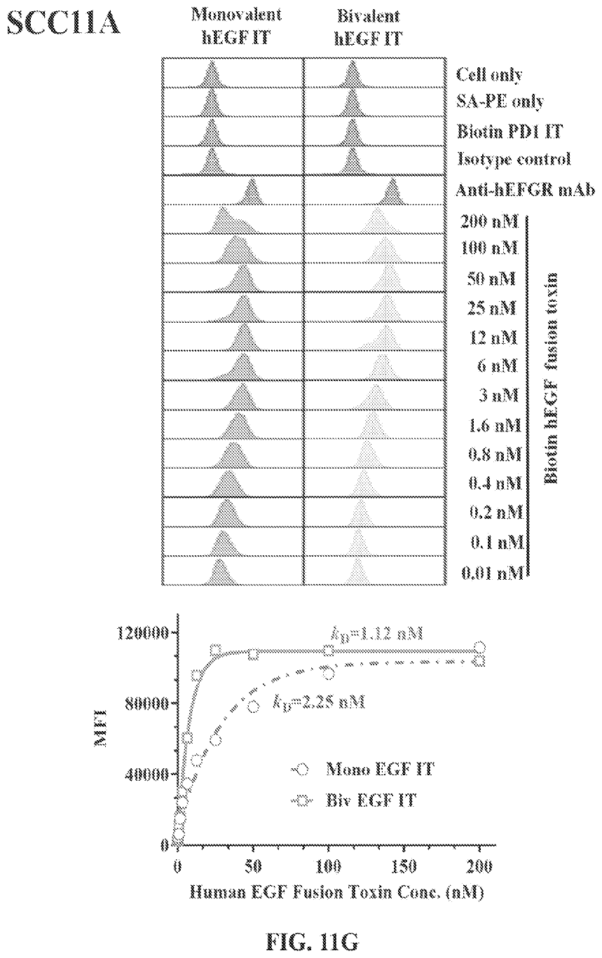

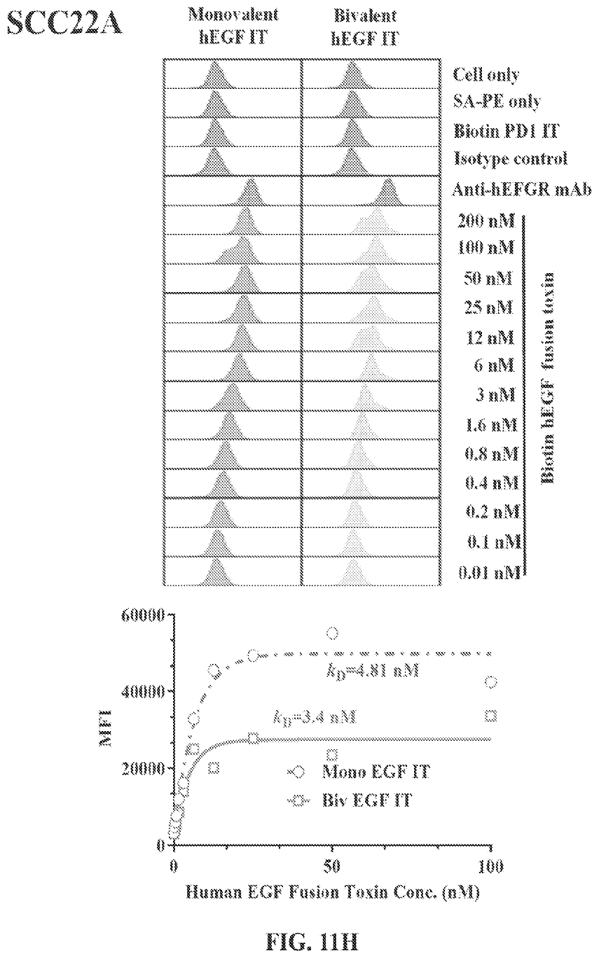

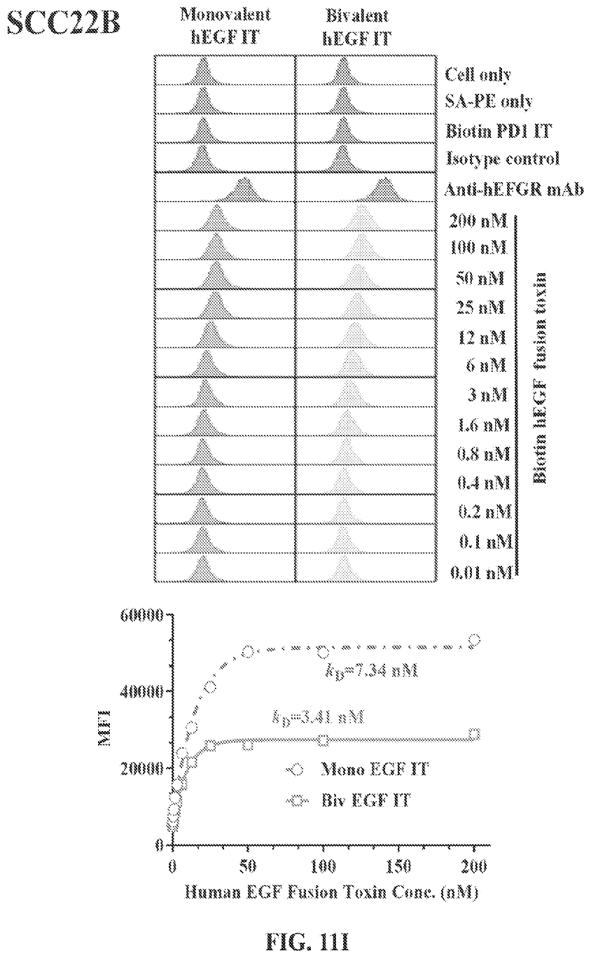

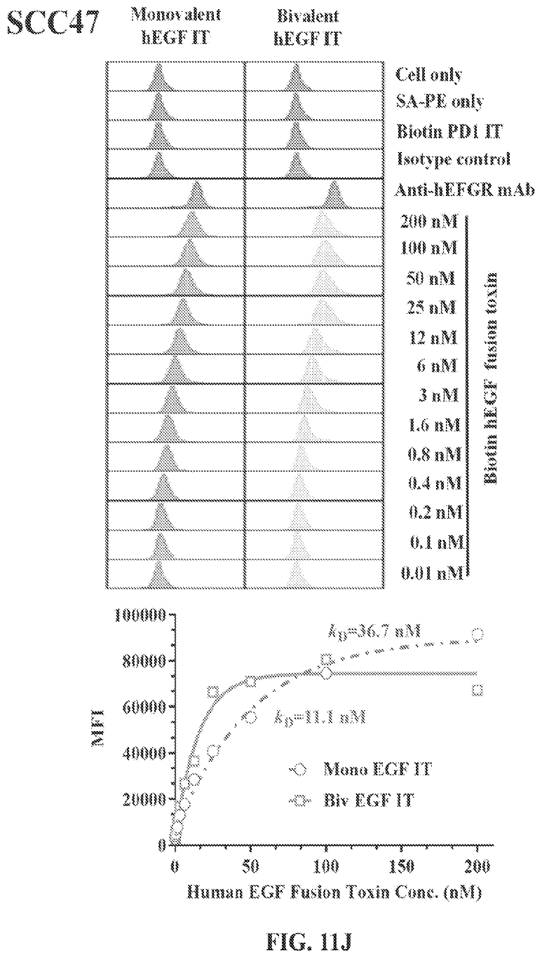

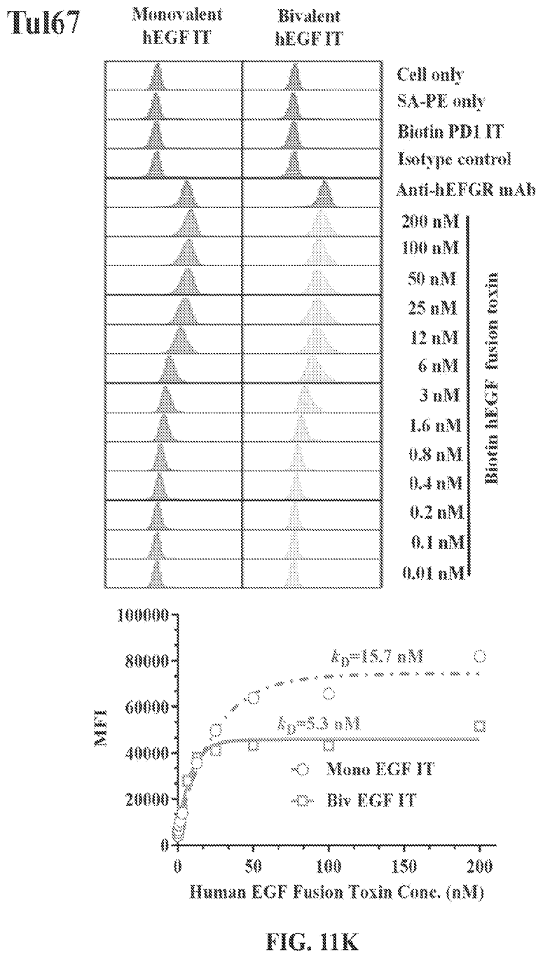

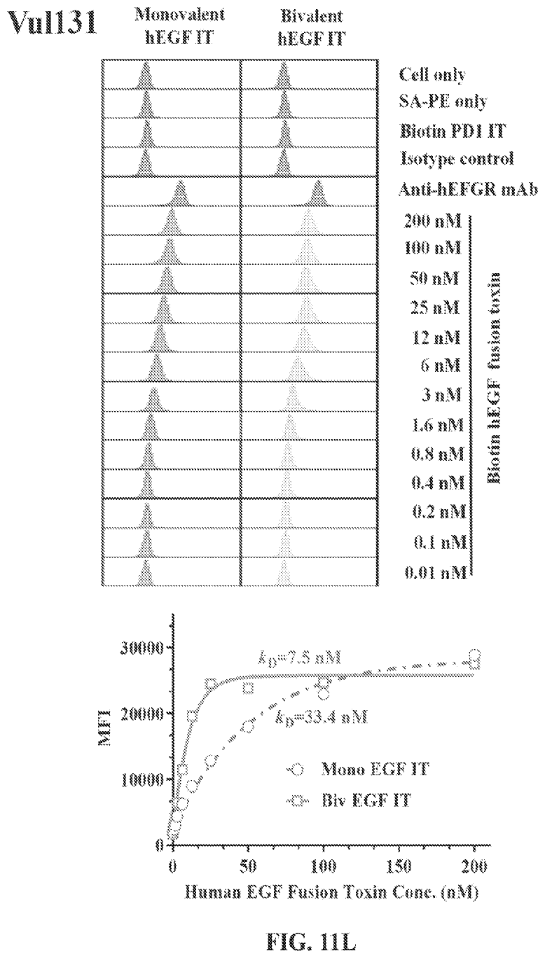

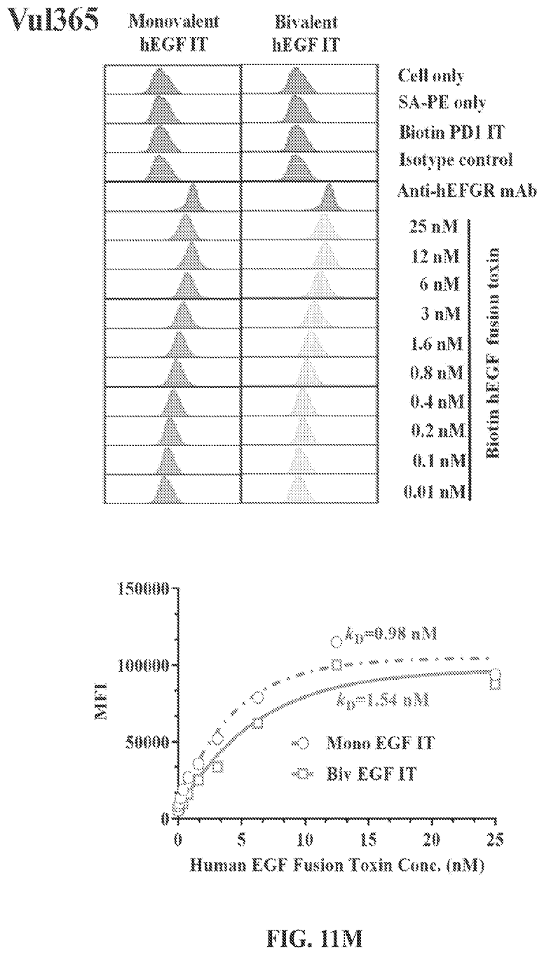

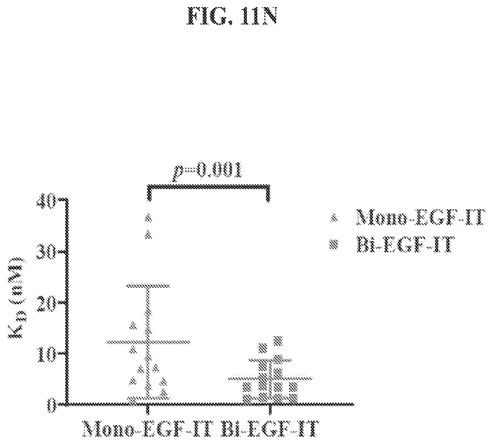

Flow cytometry binding analysis demonstrated that all examined 14 HNSCC cell lines were EGFR positive (see positive controls of FIGS. 3 and 11). As illustrated by FIGS. 3A and 3B, biotinylated mEGF-IT and bEGF-IT bound to human HNSCC Cal27 cells in a dose-dependent fashion with a K_{D} value of 18.4 nM for the monovalent fusion toxin and 12.5 nM for the bivalent fusion toxin. The biotinylated EGF fusion toxins also dose-dependently bound to all other examined HNSCC cell lines (FIG. 3, 11, and Table 4). Of note, the mean K_{D} for bi-EGF-IT (5.05 nM) was significantly lower than that for mono-EGF-IT (12.21 nM), indicating that the former had a better binding affinity (see FIG. 11N). One limitation to the flow cytometry binding assay is that the extent of biotinylation should be similar between the constructs. Otherwise, it could be hard to attribute the differences in fluorescence intensities to a change in affinity. To overcome this limitation, a blocking assay using nonbiotinylated mono-EGF-IT or bi-EGF-IT to block the binding of the anti-human EGFR mAb to the EGFR+ HNSCC cell line, Cal27, was performed (Wang et al, 2007, Bioconjug Chem 18: 944-955). The results confirmed that bi-EGF-IT bound significantly stronger to Cal27 cells compared with mono-EGF-IT (FIGS. 3C and 3D). The 14 examined HNSCC cell lines represent the main HNSCC cell lines used in the research community of the head and neck cancers.

TABLE 4
K_{D} of the human EGF fusion toxins
to human HNSCC cell lines

| | Mono-EGF IT | Bi-EGF IT |
|---|---|---|
| | (nM) | (nM) |
| Cal27 | 18.44 | 12.45 |
| Fadu | 7.13 | 6.20 |
| HN6 | 14.71 | 3.44 |
| UMSCC1 | 4.68 | 1.30 |
| UMSCC2 | 10.95 | 8.93 |
| UMSCC10A | 3.87 | 3.63 |
| UMSCC10B | 9.58 | 1.34 |
| UMSCC11A | 2.55 | 1.12 |
| UMSCC22A | 4.81 | 3.40 |
| UMSCC22B | 7.34 | 3.41 |
| UMSCC47 | 36.73 | 11.08 |
| TU167 | 15.70 | 5.30 |
| Vu1131 | 33.43 | 7.51 |
| Vu1365 | 0.98 | 1.54 |

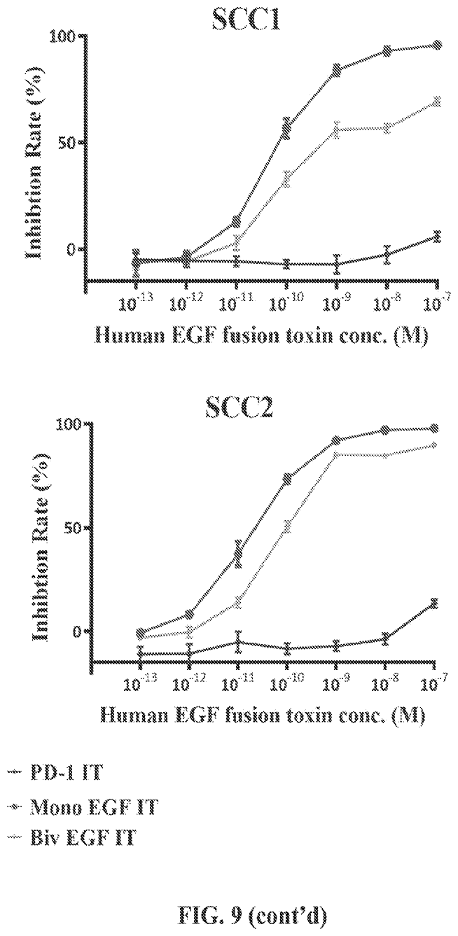

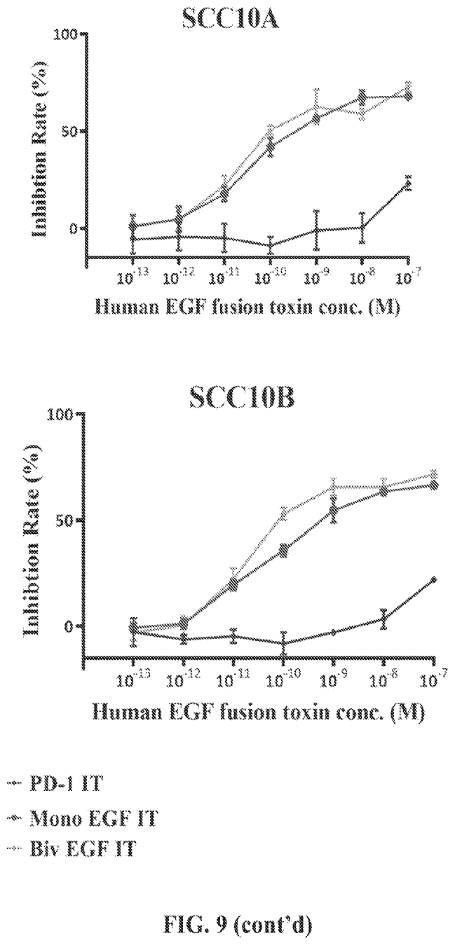

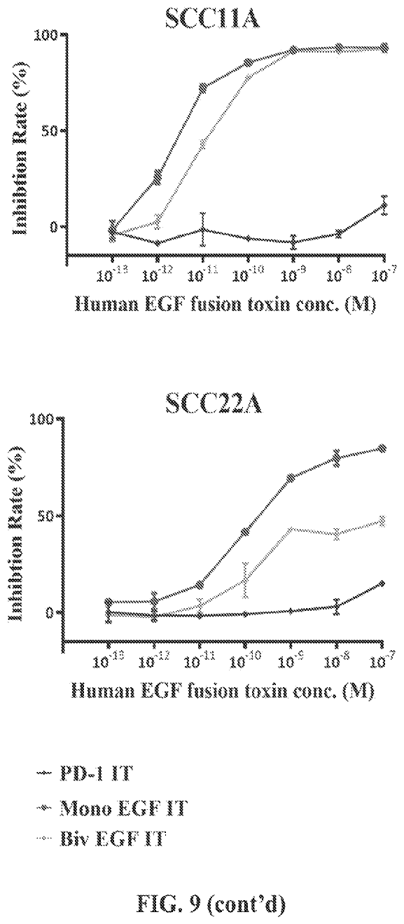

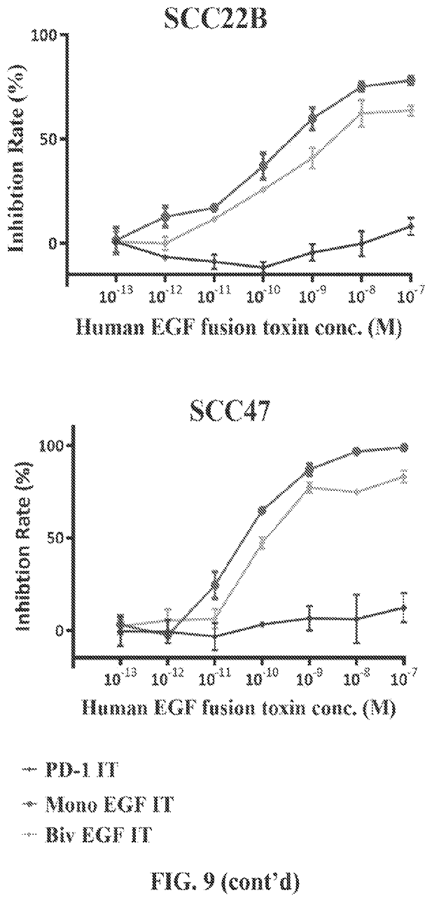

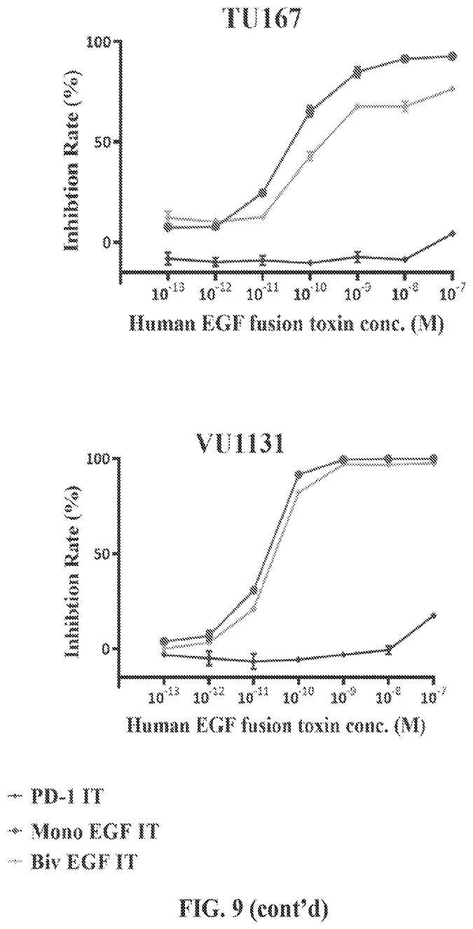

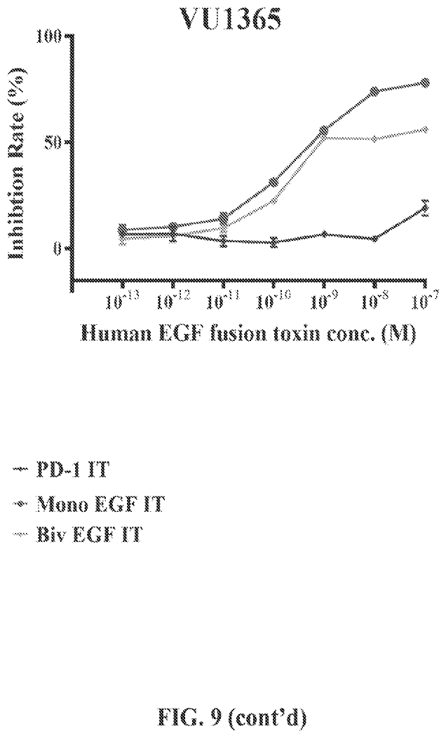

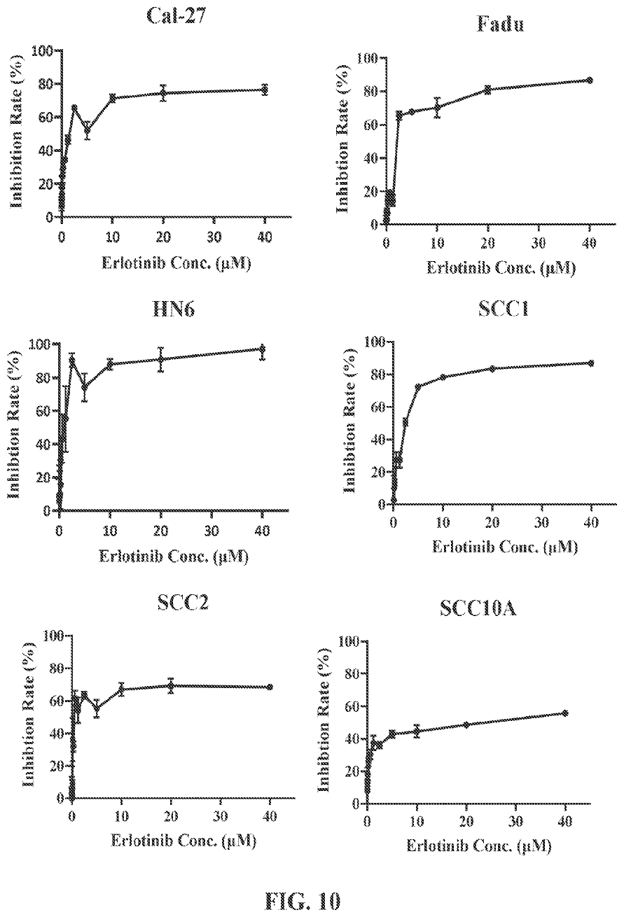

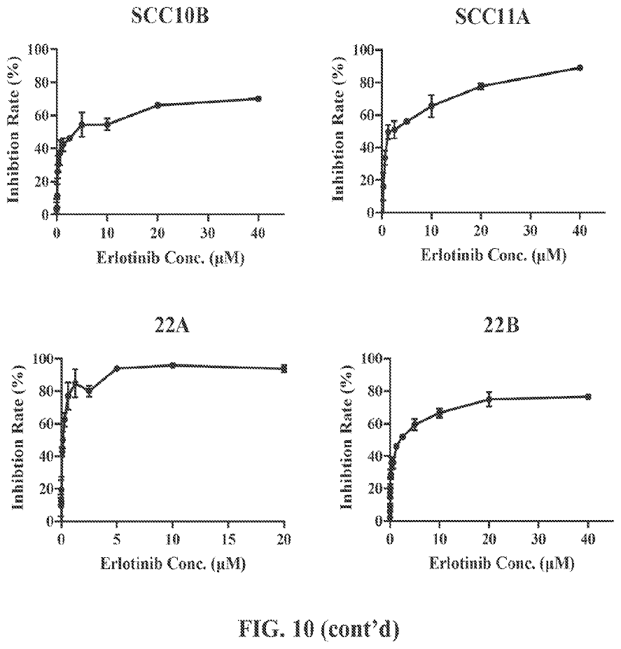

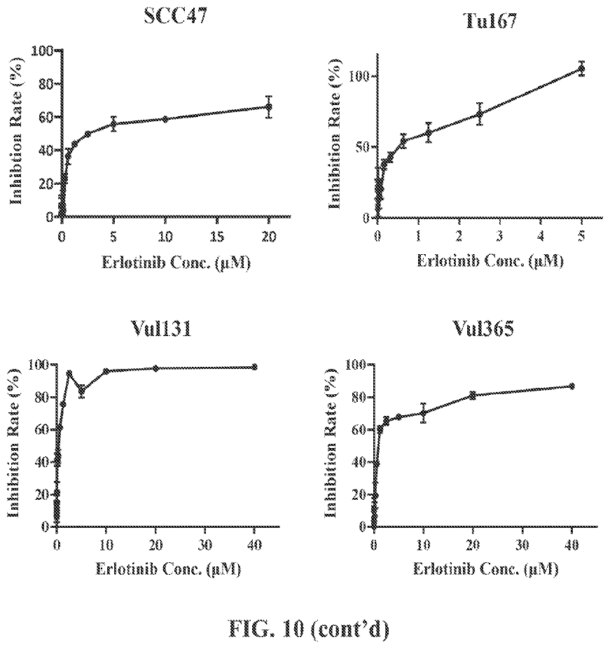

In vitro efficacy of the human EGF fusion toxins against HNSCC cell lines were assessed using CellTiter-Glo® Luminescent Cell Viability Assay. As depicted in FIG. 4, both mEGF-IT and bEGF-IT effectively depleted HNSCC Cal27 cells with an IC₅₀ value of 8.75×10⁻¹¹ M for the monovalent fusion toxin and 9.96×10⁻¹¹ M for the bivalent fusion toxin. The human EGF fusion toxins also effectively depleted all other examined HNSCC cell lines (FIG. 9 and Table 5). Positive control erlotinib was effective in vitro against all 14 examined HNSCC tumor cell lines (FIG. 10).

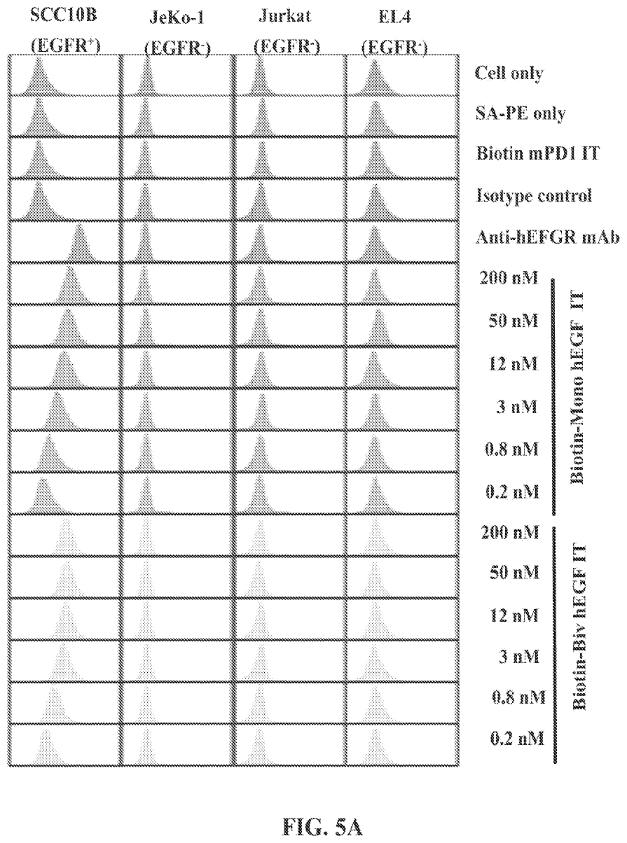

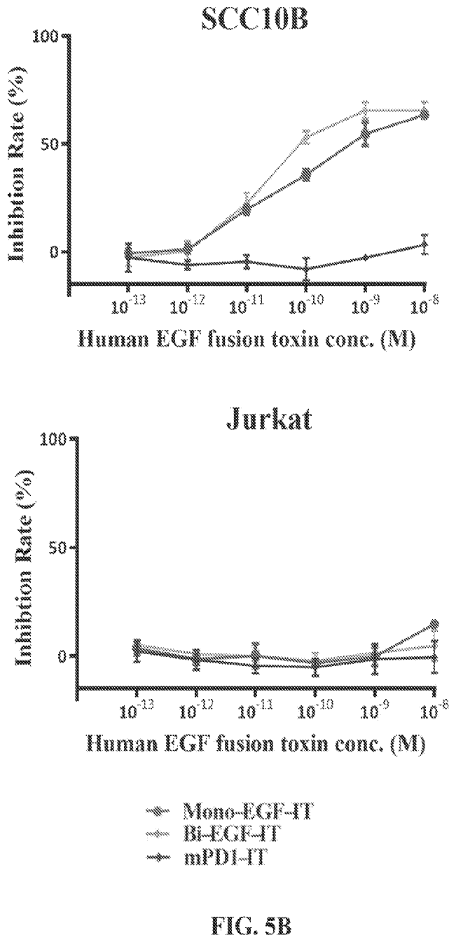

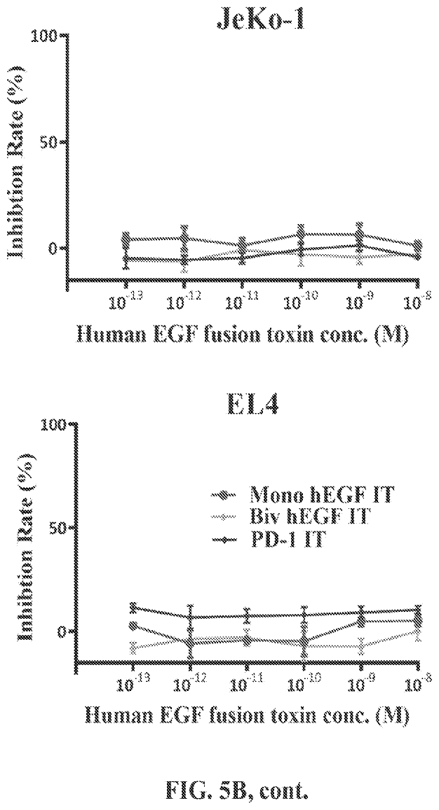

In vitro off-target analysis was also performed for three human EGFR-negative tumor cell lines including JeKo-1, Jurkat and EL-4. As depicted by FIG. 5, there was no in vitro binding or efficacy detected in the three EGFR⁻ tumor cell lines.

TABLE 5
IC₅₀ of the human EGF fusion
toxins to HNSCC cell lines

| | Mono-EGF IT | Bi-EGF IT | Erlotinib |
|---|---|---|---|
| | (M) | (M) | (μM) |
| Cal27 | 8.75E−11 | 9.96E−11 | 1.87 |
| Fadu | 8.81E−11 | 3.58E−11 | 2.83 |
| HN6 | 5.17E−11 | 1.49E−10 | 0.94 |
| UMSCC1 | 4.6E−11 | 8.48E−10 | 2.46 |
| UMSCC2 | 2.32E−11 | 1.1E−10 | 1.59 |
| UMSCC10A | 9.77E−10 | 6.37E−10 | 15.16 |
| UMSCC10B | 1.59E−09 | 4.29E−10 | 3.81 |
| UMSCC11A | 3.36E−12 | 1.48E−11 | 2.76 |
| UMSCC22A | 2.72E−10 | 4.87E−08 | 0.16 |
| UMSCC22B | 5.17E−10 | 4.54E−09 | 1.74 |
| UMSCC47 | 4.95E−11 | 2.3E−10 | 4.12 |
| TU167 | 4.82E−11 | 4.57E−10 | 0.41 |
| Vu1131 | 1.8E−11 | 2.93E−11 | 0.33 |
| Vu1365 | 7.85E−10 | 9.21E−09 | 1.54 |

### Experimental Example 3—In Vivo Efficacy Assessment of the Human EGF Fusion Toxins

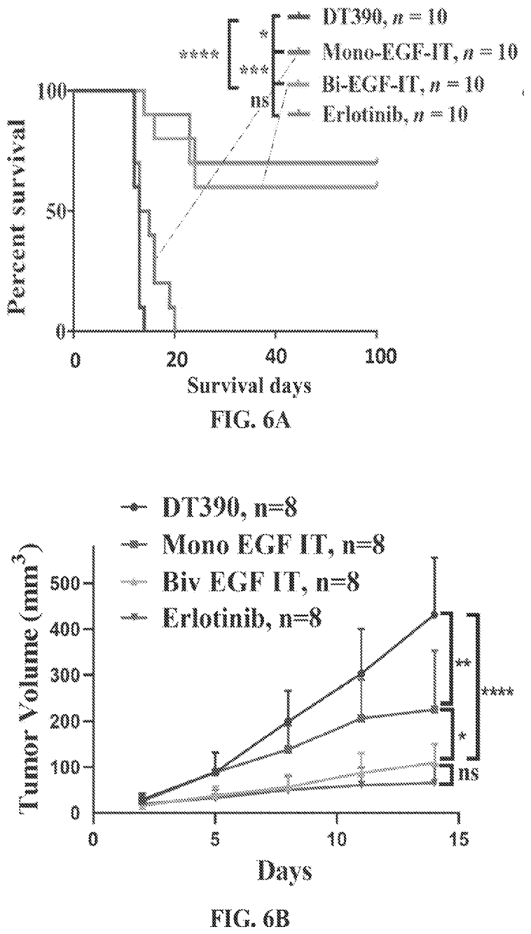

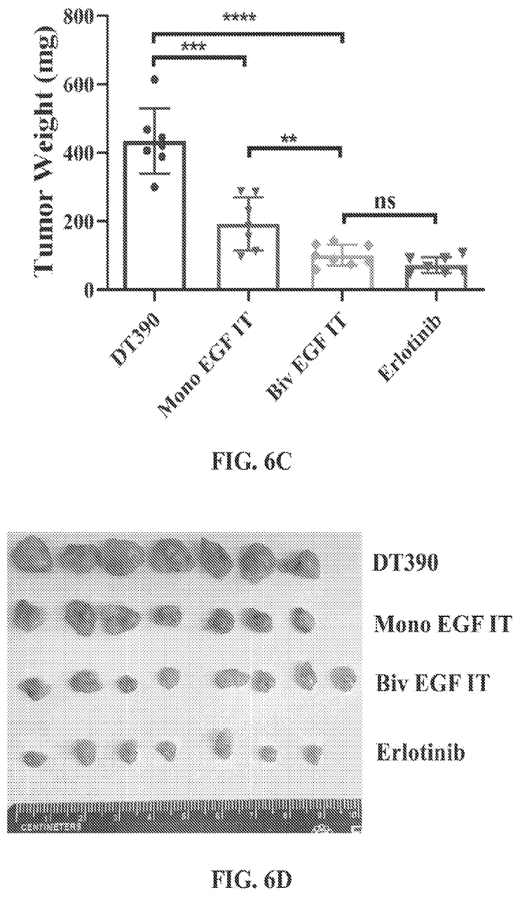

In vivo efficacy of the mEGF-IT and bEGF-IT against HNSCC was assessed using three immunodeficient tumor-bearing NSG mouse models. The first in vivo efficacy model was a flank-xenograft solid tumor-bearing NSG mouse model. Eight million Cal27 tumor cells were injected subcutaneously (SQ) into the right flank at day 0. The EGF fusion toxins were injected intraperitoneally (IP) starting on day 4 at 50 μg/kg daily for 10 consecutive days. Surprisingly, only the bivalent version markedly prolonged the median survival of the flank-xenograft tumor-bearing animals, from 14 days as seen in the negative control DT390 treatment group to 61 days in the bivalent fusion toxin treatment group. The median survival of the bivalent fusion toxin treatment group was comparable to that of the positive control erlotinib treatment group (FIG. 6A). In contrast, although it was statistically significant, the monovalent fusion toxin only prolonged the median survival slightly from 13 days as seen in the negative control DT390 treatment to 14 days with the monovalent fusion toxin treatment. The reduction of the tumor volumes, weights and sizes (FIGS. 6B-6D) were all consistent with the median survival prolongation. The bivalent fusion toxin significantly decreased tumor sizes and weights by about 80%.

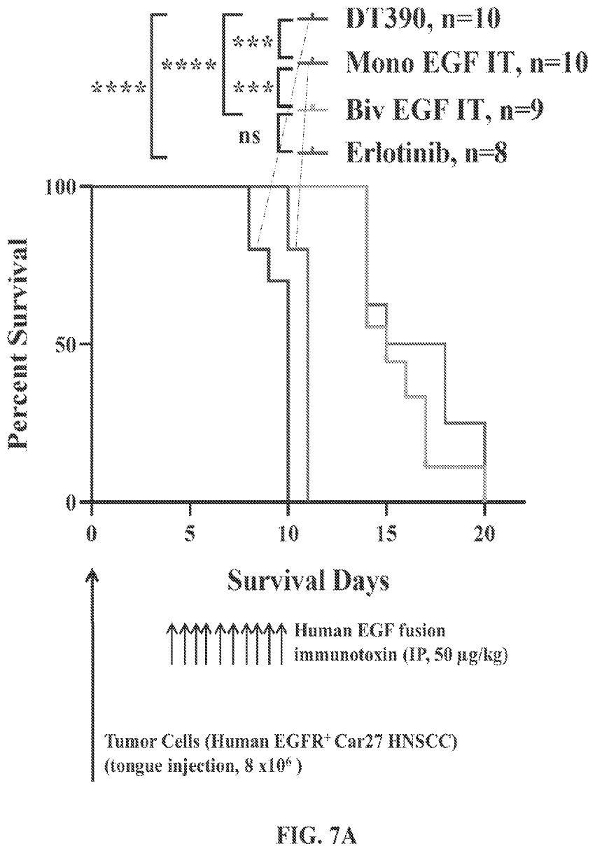

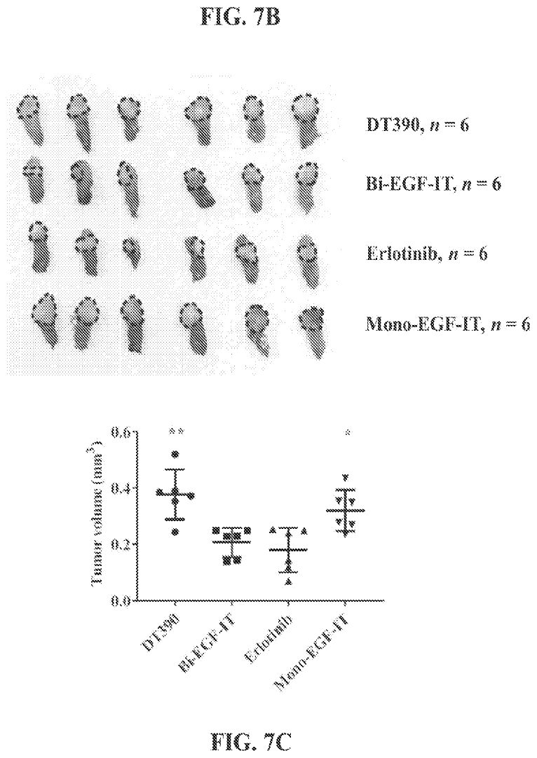

To assess the in vivo efficacy of the human EGF fusion toxins to the xenograft tumors in the head and neck area, a tongue-orthotopic tumor-bearing NSG mouse model was used as a second efficacy model. Eight million HNSCC Cal27 cells were injected into the tongue at day 0. The EGF fusion toxins were injected intraperitoneally (IP) starting on day 4 at 50 μg/kg daily for 10 consecutive days. Since the resulting tongue-orthotopic tumors severely affected the animal's eating and drinking, animals were euthanized earlier. Therefore, the median survival prolongation was not as impressive as that observed in the flank-xenograft solid tumor-bearing NSG mouse model. As depicted by FIG. 7A, the bivalent fusion toxin still significantly prolonged the median survival of the tongue-orthotopic tumor-bearing animals from 10 days as seen with the negative control DT390 treatment to 15 days with the bivalent fusion toxin treatment. See Table 6. To further characterize the effect of the EGF fusion toxins on tumor volume, the orthotopic tongue SCC model study was repeated, and the mice were euthanized 8 days after tumor cell inoculation. The tongue SCCs were significantly smaller in both the bi-EGF-IT and erlotinib groups than those from the mono-EGF-IT and DT390 control groups. See FIGS. 7B and C.

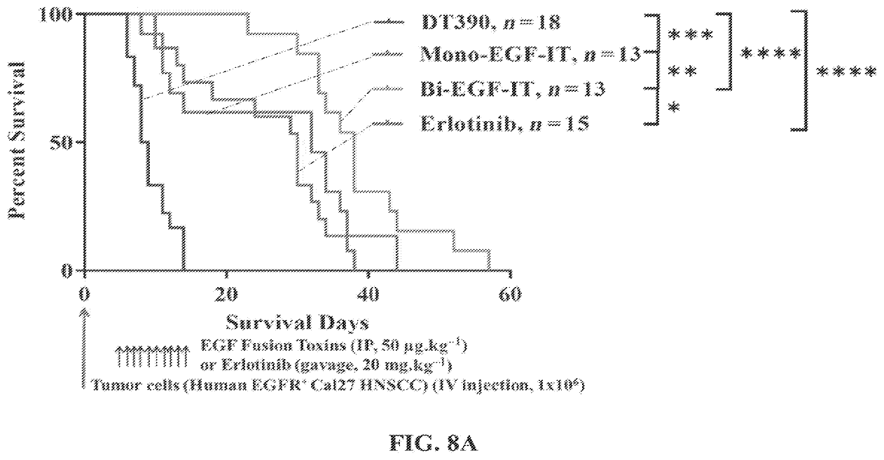

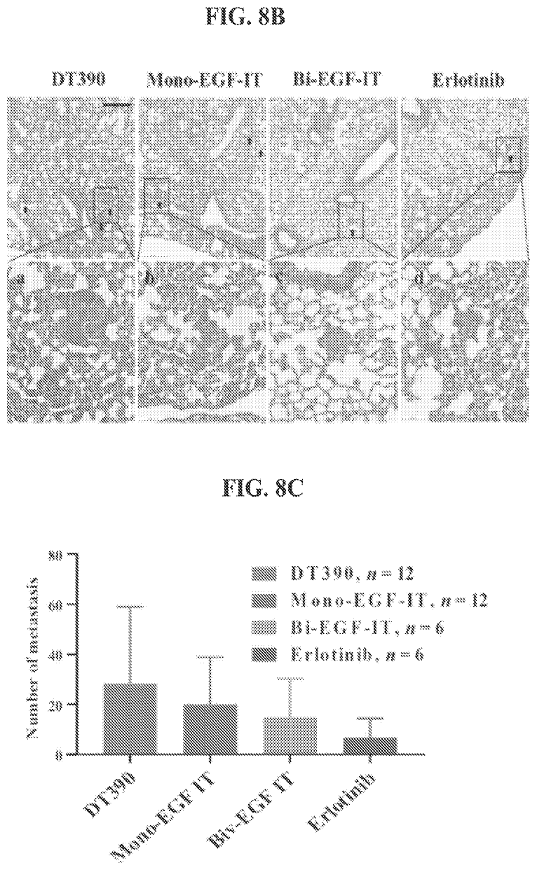

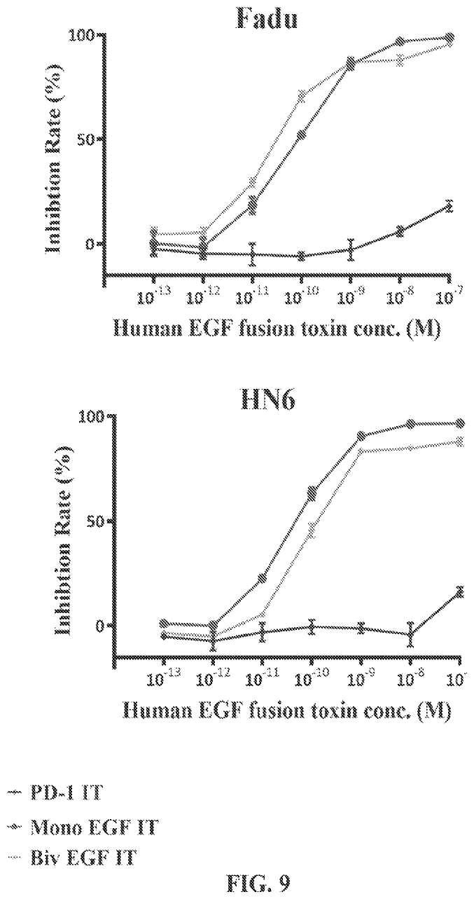

The third in vivo efficacy model was an experimental metastasis model. One million of HNSCC Cal27 tumor cells were intravenously injected via the tail vein of NSG mice at day 0. The EGF fusion toxins were injected intraperitoneally (IP) starting on day 4 at 50 μg/kg daily for 10 consecutive days. As depicted by FIG. 8A, both mEGF-IT and bEGF-IT significantly prolonged the median survival of the tumor-bearing animals from 8.5 days as seen in the negative control DT390 treatment group to 32 days in the monovalent fusion toxin treatment group and 38 days in the bivalent fusion toxin treatment group. Although both mEGF-IT and bEGF-IT were more effective than the positive control erlotinib (median survival of 30 days) in this experimental metastasis model, the bivalent fusion toxin was more effective than the monovalent fusion toxin. To further assess the effects of the fusion toxins on metastasis, the experimental metastasis study was repeated with a second cohort of NSG mice, and all mice were euthanized 10 days after tumor cell inoculation, when the first mouse in the DT390 group reached the end point. The data are depicted in FIGS. 8B and C. The numbers of metastases in the lungs of mice treated with mono-EGF-IT, bi-EGF-IT, or erlotinib were reduced compared with the DT390-negative control group. This reduction was greater in the bi-EGF-IT group than in the mono-EGF-IT group.

The median survival times for the mice in the different treatment groups in the three mouse models are summarized in Table 6.

TABLE 6
Median survival time for tumor-bearing NSG mice

| | Median survival time (days) | | | |
|---|---|---|---|---|
| Tumor Model | DT390 | Mono-EGF IT | Bi-EGF IT | Erlotinib |
| Subcutaneous | 13 | 14 | >60 | >60 |
| xenograft model | | | | |
| Tongue | 10 | 11 | 15 | 16.5 |
| orthotopic model | | | | |
| Lung metastasis | 8.5 | 32 | 38 | 30 |
| model | | | | |

Taken together, all in vivo efficacy data demonstrated that the bivalent fusion toxin was most effective at depleting HNSCC tumor cells in vivo in studied applied mouse models. The in vivo efficacy of the bivalent fusion toxin also demonstrated to be better or at least comparable to positive control erlotinib treatment.

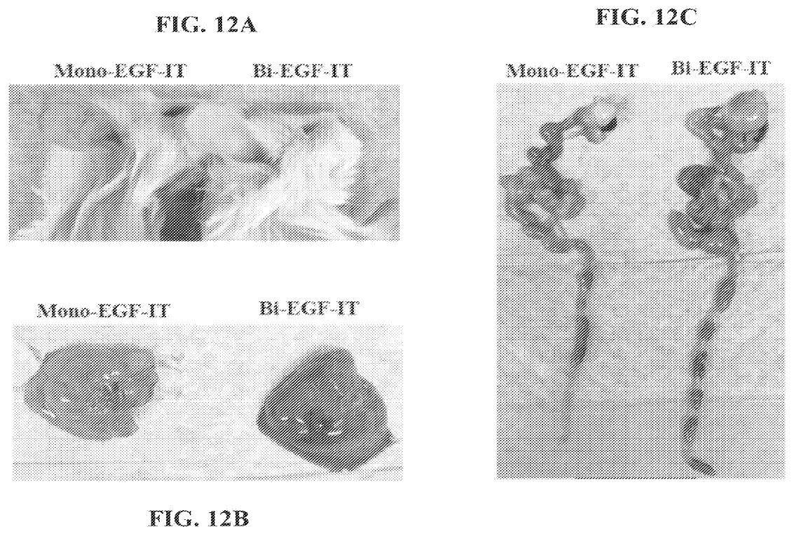

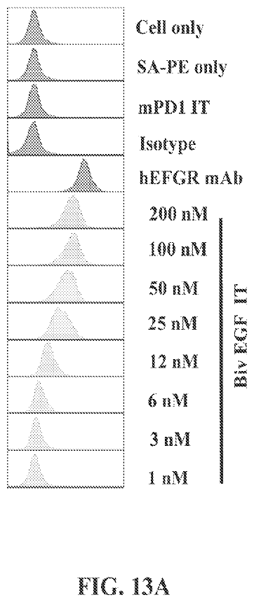

The main side effects of targeting EGFR are caused by off-target effects, because EGFR is expressed in many healthy tissues, include skin, liver, and gastrointestinal tract (Widakowich et al., (2007), Oncologist, 12, 1443-1455). Consistent with this, it was observed that mice treated with mono-EGF-IT appeared generally unhealthy and lethargic and developed skin rashes. Remarkably, however, these adverse effects were not observed with bi-EGF-IT. To further assess these toxic effects, another in vivo toxicity study in non-tumor-bearing mice was performed. NSG mice were treated with mono-EGF-IT or bi-EGF-IT, without tumor cell injection. Mice treated with mono-EGF-IT, but not bi-EGF-IT, were again generally unhealthy and lethargic with skin rashes (FIG. 12A). Necropsy of the mono-EGF-IT-treated mice 13 days after the first drug injection showed diffuse pale livers with moderate reticular patterns, suggesting lobular congestion or necrosis. In contrast, no significant abnormal findings were observed for bi-EGF-IT-treated mice (FIG. 12B). Similarly, the entire gastrointestinal tract was mildly pale with scant ingesta in the stomach or cecum in the mono-EGF-IT group compared with the normal appearance of the gastrointestinal tract in the bi-EGF-IT group (FIG. 12C). Notably, these data demonstrate that bi-EGF-IT had markedly less in vivo off-target toxicities than the monovalent EGF fusion toxin.

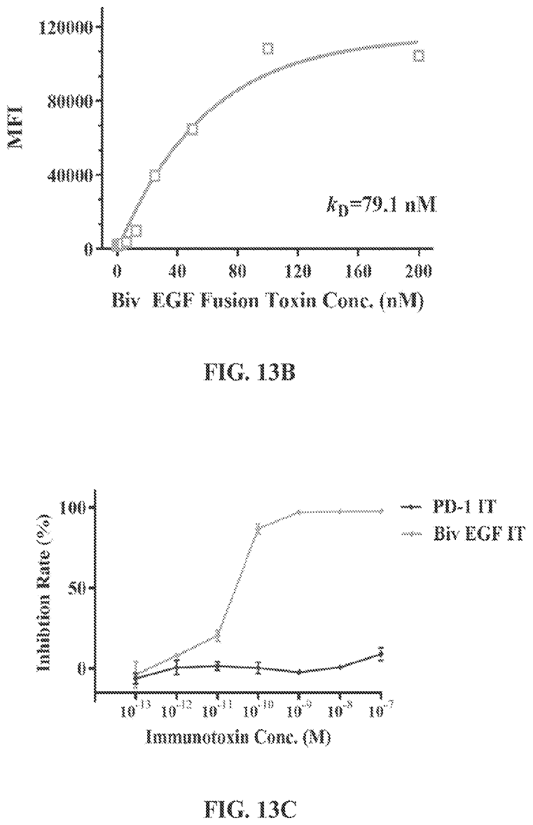

It was hypothesized that the residual tumor cells were still sensitive to the human EGF fusion toxin treatment. To confirm this hypothesis, the residual tumor cells were collected from HNSCC Cal27 flank-xenograft solid tumor-bearing mice at the study endpoint (day 14) following the treatment using the bEGF-IT at 50 μg/kg once daily for 10 consecutive days. The residual tumor cells grew very well in the same DMEM-based medium used for the parent tumor cell line Cal27. Flow cytometry binding affinity analysis demonstrated that the residual tumor cells were still EGFR positive. The biotinylated bEGF-IT bound to the residual tumor cells in a dose-dependent fashion (FIG. 13A). The K_{D} value (FIG. 13B) of the residual tumor cells was 79.1 nM. bEGF-IT also effectively depleted the residual Ca127 tumor cells in vitro in a dose-dependent manner with an IC₅₀ value of 5.62×10⁻¹¹ M (FIG. 13C), indicating that the residual tumor cells were still sensitive to the bEGF-IT treatment.

Table of Sequences

| | | SEQ |
|---|---|---|
| | | ID |
| Description | Sequence | NO |
| Human | NSDSECPLSHDGYCLHDGVCMYIEALDKYACNCVVGYIGERCQYRDLKWWE | 1 |
| EGF | LR | |
| Diphtheria | GADDVVDSSKSFVMENFSSYHGTKPGYVDSIQKGIQKPKSGTQGNYDDDWK | 2 |
| Toxin | GFYSTDNKYDAAGYSVDNENPLSGKAGGVVKVTYPGLTKVLALKVDNAETI | |
| | KKELGLSLTEPLMEQVGTEEFIKRFGDGASRVVLSLPFAEGSSSVEYINNWEQ | |
| | AKALSVELEINFETRGKRGQDAMYEYMAQACAGNRVRRSVGSSLSCINLDW | |
| | DVIRDKTKTKIESLKEHGPIKNKMSESPNKTVSEEKAKQYLEEFHQTALEHPE | |
| | LSELKTVTGTNPVFAGANYAAWAVNVAQVIDSETADNLEKTTAALSILPGIGS | |
| | VMGIADGAVHHNTEEIVAQSIALSSLMVAQAIPLVGELVDIGFAAYNFVESIIN | |
| | LFQVVHNSYNRPAYSPGHKTQPFLHDGYAVSWNTVEDSIIRTGFQGESGHDIK | |
| | ITAENTPLPIAGVLLPTIPGKLDVNKSKTHISVNGRKIRMRCRAIDGDVTFCRP | |
| | KSPVYVGNGVHANLHVAFHRSSSEKIHSNEISSDSIGVLGYQKTVDHTKVNSK | |
| | LSLFFEIKS | |
| DT₃₇₀ | GADDVVDSSKSFVMENFSSYHGTKPGYVDSIQKGIQKPKSGTQGNYDDDWK | 3 |
| | GFYSTDNKYDAAGYSVDNENPLSGKAGGVVKVTYPGLTKVLALKVDNAETI | |
| | KKELGLSLTEPLMEQVGTEEFIKRFGDGASRVVLSLPFAEGSSSVEYINNWEQ | |
| | AKALSVELEINFETRGKRGQDAMYEYMAQACAGNRVRRSVGSSLSCINLDW | |
| | DVIRDKTKTKIESLKEHGPIKNKMSESPNKTVSEEKAKQYLEEFHQTALEHPE | |
| | LSELKTVTGTNPVFAGANYAAWAVNVAQVIDSETADNLEKTTAALSILPGIGS | |
| | VMGIADGAVHHNTEEIVAQSIALSSLMVAQAIPLVGELVDIGFAAYNFVESIIN | |
| | LFQV | |
| DT₃₈₃ | GADDVVDSSKSFVMENFSSYHGTKPGYVDSIQKGIQKPKSGTQGNYDDDWK | 4 |
| | GFYSTDNKYDAAGYSVDNENPLSGKAGGVVKVTYPGLTKVLALKVDNAETI | |
| | KKELGLSLTEPLMEQVGTEEFIKRFGDGASRVVLSLPFAEGSSSVEYINNWEQ | |
| | AKALSVELEINFETRGKRGQDAMYEYMAQACAGNRVRRSVGSSLSCINLDW | |
| | DVIRDKTKTKIESLKEHGPIKNKMSESPNKTVSEEKAKQYLEEFHQTALEHPE | |
| | LSELKTVTGTNPVFAGANYAAWAVNVAQVIDSETADNLEKTTAALSILPGIGS | |
| | VMGIADGAVHHNTEEIVAQSIALSSLMVAQAIPLVGELVDIGFAAYNFVESIIN | |
| | LFQVVHNSYNRPAYSPG | |
| DT₃₈₈ | GADDVVDSSKSFVMENFSSYHGTKPGYVDSIQKGIQKPKSGTQGNYDDDWK | 5 |
| | GFYSTDNKYDAAGYSVDNENPLSGKAGGVVKVTYPGLTKVLALKVDNAETI | |
| | KKELGLSLTEPLMEQVGTEEFIKRFGDGASRVVLSLPFAEGSSSVEYINNWEQ | |
| | AKALSVELEINFETRGKRGQDAMYEYMAQACAGNRVRRSVGSSLSCINLDW | |
| | DVIRDKTKTKIESLKEHGPIKNKMSESPNKTVSEEKAKQYLEEFHQTALEHPE | |
| | LSELKTVTGTNPVFAGANYAAWAVNVAQVIDSETADNLEKTTAALSILPGIGS | |
| | VMGIADGAVHHNTEEIVAQSIALSSLMVAQAIPLVGELVDIGFAAYNFVESIIN | |
| | LFQVVHNSYNRPAYSPGHKTQP | |
| DT₃₃₉ | GADDVVDSSKSFVMENFSSYHGTKPGYVDSIQKGIQKPKSGTQGNYDDDWK | 6 |
| | GFYSTDNKYDAAGYSVDNENPLSGKAGGVVKVTYPGLTKVLALKVDNAETI | |
| | KKELGLSLTEPLMEQVGTEEFIKRFGDGASRVVLSLPFAEGSSSVEYINNWEQ | |
| | AKALSVELEINFETRGKRGQDAMYEYMAQACAGNRVRRSVGSSLSCINLDW | |
| | DVIRDKTKTKIESLKEHGPIKNKMSESPNKTVSEEKAKQYLEEFHQTALEHPE | |
| | LSELKTVTGTNPVFAGANYAAWAVNVAQVIDSETADNLEKTTAALSILPGIGS | |
| | VMGIADGAVHHNTEEIVAQSIALSSLMVAQAIPLVGELVDIGFAAYNFVESIIN | |
| | LFQVVHNSYNRPAYSPGHKTQPF | |
| DT₃₉₀ | GADDVVDSSKSFVMENFSSYHGTKPGYVDSIQKGIQKPKSGTQGNYDDDWK | 7 |
| | GFYSTDNKYDAAGYSVDNENPLSGKAGGVVKVTYPGLTKVLALKVDNAETI | |
| | KKELGLSLTEPLMEQVGTEEFIKRFGDGASRVVLSLPFAEGSSSVEYINNWEQ | |
| | AKALSVELEINFETRGKRGQDAMYEYMAQACAGNRVRRSVGSSLSCINLDW | |
| | DVIRDKTKTKIESLKEHGPIKNKMSESPNKTVSEEKAKQYLEEFHQTALEHPE | |
| | LSELKTVTGTNPVFAGANYAAWAVNVAQVIDSETADNLEKTTAALSILPGIGS | |
| | VMGIADGAVHHNTEEIVAQSIALSSLMVAQAIPLVGELVDIGFAAYNFVESIIN | |
| | LFQVVHNSYNRPAYSPGHKTQPFL | |
| G₄S linker | GGGGS | 8 |
| (G₄S)₃ | GGGGSGGGGSGGGGS | 9 |
| linker | | |
| Bivalent | AGADDVVDSSKSFVMENFASYHGTKPGYVDSIQKGIQKPKSGTQGNYDDDW | 10 |
| human | KGFYSTDNKYDAAGYSVDNENPLSGKAGGVVKVTYPGLTKVLALKVDNAE | |
| EGF-DT390 | TIKKELGLSLTEPLMEQVGTEEFIKRFGDGASRVVLSLPFAEGSSSVEYINNWE | |
| fusion toxin | QAKALSVELEINFETRGKRGQDAMYEYMAQACAGNRVRRSVGSSLSCINLD | |
| | WDVIRDKTKTKIESLKEHGPIKNKMSESPAKTVSEEKAKQYLEEFHQTALEHP | |
| | ELSELKTVTGTNPVFAGANYAAWAVNVAQVIDSETADNLEKTTAALSILPGIG | |
| | SVMGIADGAVHHNTEEIVAQSIALSSLMVAQAIPLVGELVDIGFAAYNFVESII | |
| | NLFQVVHNSYNRPAYSPGHKTQPFLPWGGGGSNSDSECPLSHDGYCLHDGV | |
| | CMYIEALDKYACNCVVGYIGERCQYRDLKWWELRGGGGSGGGGSGGGGSN | |
| | SDSECPLSHDGYCLHDGVCMYIEALDKYACNCVVGYIGERCQYRDLKWWEL | |
| | RHHHHHH | |
| hEGF-Nco | CATGCCATGGGGTGGTGGTGGTTCTAACTCTGACTCCGAGTGTCCA | 11 |
| primer | | |
| hEGF- | CGGGGATCCACCACCACCAGAACCACCACCACCTCTCAACTCCCACCACT | 12 |
| Baml | TCAA | |
| primer | | |
| hEGF- | CGGGGATCCGGTGGTGGTGGTTCTAACTCTGACTCCGAGTGTCCA | 13 |
| Bam2 | | |
| primer | | |
| hEGF-Eco | CCGGAATTCTTAGTGGTGGTGGTGGTGGTGTCTCAACTCCCACCACTTCAA | 14 |
| primer | | |
| Monovalent | AGADDVVDSSKSFVMENFASYHGTKPGYVDSIQKGIQKPKSGTQGNYDDDW | 15 |
| human | KGFYSTDNKYDAAGYSVDNENPLSGKAGGVVKVTYPGLTKVLALKVDNAETI | |
| EGF-DT₃₉₀ | KKELGLSLTEPLMEQVGTEEFIKRFGDGASRVVLSLPFAEGSSSVEYINNWE | |
| fusion toxin | QAKALSVELEINFETRGKRGQDAMYEYMAQACAGNRVRRSVGSSLSCINLDW | |
| | DVIRDKTKTKIESLKEHGPIKNKMSESPAKTVSEEKAKQYLEEFHQTALEHP | |
| | ELSELKTVTGTNPVFAGANYAAWAVNVAQVIDSETADNLEKTTAALSILPGI | |
| | SGVMGIADGAVHHNTEEIVAQSIALSSLMVAQAIPLVGELVDIGFAAYNFVE | |
| | SIINLFQVVHNSYNRPAYSPGHKTQPFLPWGGGGSNSDSECPLSHDGYCLHDGV | |
| | CMYIEALDKYACNCVVGYIGERCQYRDLKWWELRHHHHHH | |
| linker can | GGGGSGGGGSGGGGSGGGGSGGGGS | 16 |
| be (G₄S)ₙ, | | |
| where n = | | |
| 1-5 | | |
| 6x His tag | HHHHHH | 17 |

1. A bivalent fusion toxin comprising:
two epidermal growth factor (EGF) domains;
a diphtheria toxin (DT) domain; and
at least one linker.

2. The bivalent fusion toxin of claim 1, wherein the two epidermal growth factor domains each individually consist of human EGF or an EGFR-binding fragment thereof.

3. The bivalent fusion toxin of claim 2, wherein human EGF has an amino acid sequence having at least 95% identity to SEQ ID NO: 1.

4. The bivalent fusion toxin of claim 1, wherein the DT domain is selected from the group consisting of: DT₃₁₀ (SEQ ID NO: 3), DT₃₈₃ (SEQ ID NO: 4), DT₃₈₈ (SEQ ID NO: 5), DT₃₈₈ (SEQ ID NO: 6), and DT₃₉₀ (SEQ ID NO: 7).

5. The bivalent fusion toxin of claim 1, wherein the DT domain is DT₃₉₀ (SEQ ID NO: 7).

6. The bivalent fusion toxin of claim 1, wherein the at least one linker includes at least one of G₄S (SEQ ID NO: 8) and (G₄S)₃ (SEQ ID NO: 9).

7. The bivalent fusion toxin of claim 1, wherein the bivalent fusion toxin includes a first linker between the DT domain and a first EGF domain, and a second linker between the first EGF domain and a second EGF domain.

8. The bivalent fusion toxin of claim 7, wherein the first linker is G₄S (SEQ ID NO: 8) and the second linker is (G₄S)₃ (SEQ ID NO: 9).

9. The bivalent fusion toxin of claim 1, wherein the bivalent fusion toxin comprises an amino acid sequence having at least 90% identity to SEQ ID NO: 10.

10. A nucleic acid molecule encoding the bivalent fusion toxin of claim 1.

11. The nucleic acid molecule of claim 10, wherein the nucleic acid molecule is codon optimized.

12. An expression vector comprising the nucleic acid molecule of claim 10.

13. An isolated cell comprising the expression vector of claim 12.

14. A pharmaceutical composition comprising the bivalent fusion toxin of claim 1 and a pharmaceutically acceptable carrier.

15. A method of treating an epidermal growth factor receptor (EGFR)-positive cancer in a patient in need thereof, comprising administering to the patient a therapeutically effective amount of the pharmaceutical composition of claim 14.

16. The method of claim 15, wherein the EGFR-positive cancer is an EGFR-positive neck and head squamous cell carcinoma (HNSCC).

17. The method of claim 15, wherein the EGFR-positive cancer is non-responsive to or recurring following standard of care treatment.

18. The method of claim 17, wherein the EGFR-positive cancer is an EGFR-positive HNSCC and the standard of care treatment includes administration of erlotinib to the patient.

19. The method of claim 15, wherein EGFRs of EGFR− positive cancer cells include at least one amino acid mutation.

20. The method of claim 19, wherein the at least one amino acid mutation includes T790M.


SEQUENCE LISTING (17 sequences)

SEQ ID NO: 1 - protein, 53 aa, Homo sapiens
NSDSECPLSHDGYCLHDGVCMYIEALDKYACNCVVGYIGERCQYRDLKWWELR

SEQ ID NO: 2 - protein, 535 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 3 - protein, 370 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQV

SEQ ID NO: 4 - protein, 383 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPG

SEQ ID NO: 5 - protein, 388 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQP

SEQ ID NO: 6 - protein, 389 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPF

SEQ ID NO: 7 - protein, 390 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFL

SEQ ID NO: 8 - protein, 5 aa, Artificial Sequence
  Description of Artificial Sequence Synthetic peptide
GGGGS

SEQ ID NO: 9 - protein, 15 aa, Artificial Sequence
  Description of Artificial Sequence Synthetic peptide
GGGGSGGGGSGGGGS

SEQ ID NO: 10 - protein, 525 aa, Artificial Sequence
  Description of Artificial Sequence Synthetic polypeptide
AGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWGGGGSNSDSECPLSHDGYCLHDGVCMYIEALDKYACNCVVGYIGERCQYRDLKWWELRGGGGSGGGGSGGGGSNSDSECPLSHDGYCLHDGVCMYIEALDKYACNCVVGYIGERCQYRDLKWWELRHHHHHH

SEQ ID NO: 11 - DNA, 46 bp, Artificial Sequence
  Description of Artificial Sequence Synthetic primer
catgccatggggtggtggtggttctaactctgactccgagtgtcca

SEQ ID NO: 12 - DNA, 54 bp, Artificial Sequence
  Description of Artificial Sequence Synthetic primer
cggggatccaccaccaccagaaccaccaccacctctcaactcccaccacttcaa

SEQ ID NO: 13 - DNA, 45 bp, Artificial Sequence
  Description of Artificial Sequence Synthetic primer
cggggatccggtggtggtggttctaactctgactccgagtgtcca

SEQ ID NO: 14 - DNA, 51 bp, Artificial Sequence
  Description of Artificial Sequence Synthetic primer
ccggaattcttagtggtggtggtggtggtgtctcaactcccaccacttcaa

SEQ ID NO: 15 - protein, 457 aa, Artificial Sequence
  Description of Artificial Sequence Synthetic polypeptide
AGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWGGGGSNSDSECPLSHDGYCLHDGVCMYIEALDKYACNCVVGYIGERCQYRDLKWWELRHHHHHH

SEQ ID NO: 16 - protein, 25 aa, Artificial Sequence
  Description of Artificial Sequence Synthetic peptide
  SITE (1)..(25): This sequence may encompass 1-5 “Gly Gly Gly Gly Ser” repeating units
GGGGSGGGGSGGGGSGGGGSGGGGS

SEQ ID NO: 17 - protein, 6 aa, Artificial Sequence
  Description of Artificial Sequence Synthetic 6xHis tag
HHHHHH